(19) United States
(12) Patent Application Publication
(10) Pub. No.: US 2026/0216047 A1
(43) Pub. Date: Jul. 30, 2026

(54) HAIR CARE COMPOSITION, COMPRISING A CROSSLINKED POLYESTER COMPONENT

(71) Applicant: Momentive Performance Materials Inc., Niskayuna, NY (US)

(72) Inventors: Sarah Peeler BLACK, New York, NY (US); Xu QIN, Northvale, NJ (US); Roxanne Louisa Jean SMITH, Lechlade (GB); Rebecca ROSE, Witney (GB); Polina TSUPKO, Bronx, NY (US); Amar PAWAR, Briarcliff Manor, NY (US); Benjamin FALK, Yorktown Heights, NY (US)

(21) Appl. No.: 19/427,721

(22) Filed: Dec. 19, 2025

Related U.S. Application Data
(60) Provisional application No. 63/736,795, filed on Dec. 20, 2024.

(51) Int. Cl. A61K 8/85; A61K 8/04; A61K 8/34; A61K 8/362; A61K 8/368; A61K 8/44; A61K 8/84; A61Q 5/00; A61Q 5/12

(52) U.S. Cl. CPC A61K 8/85; A61K 8/042; A61K 8/342; A61K 8/345; A61K 8/362; A61K 8/368; A61K 8/44; A61K 8/84; A61Q 5/002; A61Q 5/12

(57) ABSTRACT

The present invention relates to a hair care composition, comprising at least one crosslinked polyester component (A), optionally one or more hair care active compounds (B), optionally one or more cosmetically acceptable carriers (C), and optionally one or more other components commonly used in the cosmetic field (D), where components (A), (B), (C) and (D) are each different from each other. The invention further relates to the use of a crosslinked polyester component for the preparation of hair care compositions and for achieving certain effects of the hair care compositions.

The present invention relates to a hair care composition, comprising at least one crosslinked polyester component (A), optionally one or more hair care active compounds (B), optionally one or more cosmetically acceptable carriers (C), and optionally one or more other components commonly used in the cosmetic field (D), where components (A), (B), (C) and (D) are each different from each other. The invention further relates to the use of a crosslinked polyester component for the preparation of hair care compositions and for achieving certain effects of the hair care compositions.

### Statement of the Problem

Crosslinked polymers are added to manipulate the sensory, texture, rheology, and optical performance of a variety of cosmetic products. Traditional silicone elastomers have limited versatility in terms of compatibility with certain ingredients. Therefore, although the performance of silicone elastomers is unparalleled, there is a demand for alternatives to silicone elastomers for use in hair care formulations and other personal care products.

### STATE OF THE ART

The personal care industry thrives on being able to deliver multiple performance products based on mixtures of several components, with each having performance characteristics important to or desirable in the final formulation. Silicone gels are commonly added in a variety of personal care formulations such as hair care formulations to enhance their aesthetics with respect to sensory, texture, rheology, and optical performance. See for example, U.S. Pat. Nos. 5,654,362; 5,760,116; 6,423,322; and 5,811,487.

Crosslinked polymers are added to manipulate the sensory, texture, rheology, and optical performance of a variety of cosmetic products. Silicone elastomers are particularly important because they can form elastic particles of three-dimensional polymeric dimethicone and provide a beneficial sensory, texture, and optical effect to cosmetic products. However, traditional silicone elastomers have limited versatility in terms of compatibility with polar solvents or emollients such as hydrocarbon oils, plant-based oils, glycerin, and water. Therefore, there is a demand for alternatives to the use of silicone elastomers in hair compositions.

US20210059924A1 disclosed a polyurethane elastomeric rubber composition containing a bio-based polyol cross-linked with a bio-based isocyanate. The cross-linked polyurethane elastomer rubber is in further aspect of the invention included in a gel after being milled in the presence of a bio-based emollient or mixture of bio-based emollients. The polyurethane elastomeric gel has good compatibility with cosmetic and natural oils and can be used as a gelling agent for these oils among other desirable cosmetic formulary roles.

Polyesters are a class of compounds that contain an ester functional group in their polymer chain. The ester group can be hydrolyzed when treated with certain biological catalysts or certain mixed cultures of microorganisms which renders a large number of polyesters biodegradable. There is a growing interest in recent years to design and develop biobased polyesters from renewable resources such as emollients, emulsifiers, film formers, or other functional ingredients for personal care applications. See for example, U.S. Pat. Nos. 8,414,906; 9,334,358; 6,540,987; and 7,820,758. However, no polyester elastomer or polyester elastomer gel has yet been reported that provides multiple benefits to consumers as a beneficial substitute to silicone gels in hair care compositions, leaving alone that polyester gels as such could be effective as hair care actives to achieve certain effects on the hairs.

### DETAILED DESCRIPTION OF THE INVENTION

Surprisingly it has been found that a hair care composition comprising at least one crosslinked polyester component (A) provides beneficial hair care effects even without the use of silicone-based hair care active compounds, such as hair frizz control, hair smoothing, hair detangling, hair friction reduction, hair combing facilitating and others as will be shown below.

The present invention thus provides a hair care composition, comprising:
(A) at least one crosslinked polyester component, comprising a crosslinked polyester which is reaction product of:
   (i) at least one compound selected from a polycarboxylic acid, a poly-carboxylic acid ester, and combinations thereof,
   (ii) at least one polyol, and
   (iii) optionally one or more monofunctional component selected from
      (a) a mono-carboxylic acid; and
      (b) a mono-alcohol.

In an embodiment the hair care composition comprises:
(A) at least one crosslinked polyester component, comprising a crosslinked polyester which is reaction product of:
   (i) at least one compound selected from a polycarboxylic acid, a poly-carboxylic acid ester, and combinations thereof,
   (ii) at least one polyol, and
   (iii) (a) at least one mono-carboxylic acid; and optionally (iii) (b) one or more a mono-alcohol.

In an embodiment the hair care composition further comprises:
(B) optionally one or more hair care active compounds, different from components (A), (C) or (D),
(C) at least one cosmetically acceptable carrier different from components (A), (B) or (D), and
(D) optionally one or more other components commonly used in the cosmetic field, different from components (A), (B) or (C).

In an embodiment the hair care composition are used to treat the hair and/or the scalp in particular of humans, in particular for cosmetic purposes.

In an embodiment the hair care composition is selected from the group consisting of shampoo, including conditioning shampoo, such as reconstructive shampoo, scalp care compositions, hair conditioning compositions, hair frizz controlling compositions, hair smoothing compositions, such as dry hair smoothing compositions, hair compositions, hair friction reducing compositions, hair combing facilitating compositions, hair repairing compositions, hair split ends reducing compositions, hair volume controlling compositions, such as hair volume- or bulk surface-reducing compositions, hair thickness-increasing compositions, hair strengthening compositions, hair colour protection compositions, hair shine enhancing compositions, hair moisturizing and hydrating compositions, hair deep conditioning compositions, as hair hold conditioning compositions, hair curl defining compositions, hair cleansing conditioning compositions, preferably hair frizz controlling compositions and hair volume controlling compositions, such as hair volume- or bulk surface-reducing compositions.

In an embodiment the hair care composition is selected from the group of leave-in conditioners, rinse-out or rinse-through conditioners, hair styling compositions, such as hair sprays, gels, creams, mousses, lotions, pastes, pomades, waxes and foams, and hair masks.

As will be described in more detail below the crosslinked polyester component (A) (sometimes called polyester elastomer) can be used as dry powder or as a gel which is obtained by swelling the crosslinked polyester e.g. under shear force in a solvent (such as a low molecular weight emollient) to form a uniform polyester gel or paste having a wide viscosity range. The gel is usually a semi-solid colloid formed by suspending the crosslinked polyester in the solvent or emollient such as a triglyceride solvent, a mono-ester solvent, a di-ester solvent, a citrate ester solvent, an ether solvent, a carbonate solvent, a hydrocarbon solvent, a silicone solvent, or a combination thereof, each as described more specifically below. The solid particles of the crosslinked polyester in the gel are supposed to form a three-dimensional network that captures and holds the solvent. The crosslinked polyester gel as component (A) is usually a stable, jelly-like substance that is soft and flexible. These crosslinked polyester components (A) in particular provided as gels in a solvent are expected to deliver superior performance benefits such as improved sensory, structuring, and rheological performance also in hair care compositions.

In an aspect, the crosslinked polyester component (A) is a reaction product of: compound (i) selected from the group consisting of
(1) one or more poly-carboxylic acids of formula (I)

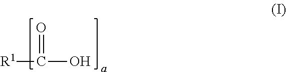

wherein
   R¹ is selected from the group consisting of C₂-C₅₂ alkyl group, C₂-C₅₂ heteroalkyl group, C₂-C₅₂ alkene group, C₂-C₅₂ heteroalkene group, C₃-C₅₂ cyclic group, or C₂-C₅₂ heterocyclic group, and
   a is an integer from 2 to 10, or
(2) one or more carboxylic acid esters of formula (II)

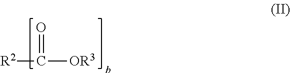

wherein
   R² is selected from the group consisting of C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
   R³ is selected from the group consisting of C1-C22 alkyl group, C2-C22 alkylene group, or C3-C22 cyclic group, and
   b is an integer from 2 to 10, and
(3) a combination (1) and (2) thereof, compound (ii) selected from one or more polyols of formula (III)

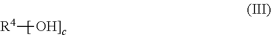

wherein
   R⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
   c is an integer from 2 to 10, and
   optional compound (iii) selected from the group consisting of
(a) one or more mono-carboxylic acids of formula (IV)

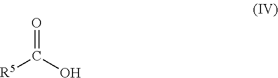

wherein
   R⁵ is C2-C52 alkyl group, C2-C52 heteroalkyl group, C2-C52 alkene group, C2-C52 heteroalkene group, C3-C52 cyclic group, or C2-C52 heterocyclic group,
(b) one or more mono-alcohols of formula (V)

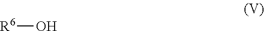

wherein
   R⁶ is C2-C52 alkyl group, C2-C52 heteroalkyl group, C2-C52 alkene group, C2-C52 heteroalkene group, C3-C52 cyclic group, or C2-C52 heterocyclic group.

In an aspect, the method of preparing a polyester elastomer (A) comprising reacting:
(i) at least one poly-carboxylic acid, at least one poly-carboxylic acid ester, or a combination thereof, and
(ii) at least one polyol; and
(iii) optionally one or more mono-carboxylic acids; and
(iv) optionally one or more mono-alcohols.

In an aspect, the crosslinked polyester elastomer (A) is the reaction product of:
(i) at least one polycarboxylic acid, at least one polycarboxylic acid ester, or a combination thereof, and
(ii) at least one polyol; and
(iii) at least one mono-carboxylic acid; and/or
(iv) at least one mono-alcohol.

In an aspect, the crosslinked polyester component (A) is reaction product of:
(i) one or more poly-carboxylic acids selected from di-carboxylic acids, tri-carboxylic acids and combinations thereof;
(ii) one or more polyols selected from diols, triols and combinations thereof, and
(iii) optionally one or more monofunctional carboxylic acids.

In an aspect, the crosslinked polyester (A) is reaction product of:
(i) one or more dicarboxylic acids
(ii) one or more triols, and
(iii) optionally one or more monofunctional carboxylic acids.

In an aspect, the preparation of crosslinked polyester component (A) is without solvent or emollient. In an aspect, the preparation of crosslinked polyester component (A) is with solvent or emollient as defined herein.

In an aspect, crosslinked polyester component (A) is only comprised of polyester. In an aspect, polyester elastomer is only comprised of crosslinked polyester. In an aspect, crosslinked polyester component (A) is comprised of crosslinked polyester and non-crosslinked polyester.

In an aspect, crosslinked polyester component (A) is comprised of polyester and solvent or emollient. In an aspect, crosslinked polyester component (A) is comprised of crosslinked polyester and solvent or emollient. In an aspect, the crosslinked polyester component (A) is comprised of crosslinked polyester, non-crosslinked polyester, and solvent or emollient.

In an aspect, the crosslinked polyester component (A) is a powder.

The crosslinked polyester component (A) forms crosslinked polymer networks. As is well-known to a skilled person in the art such crosslinked polymer are not (completely) soluble and certain methods are available to characterize them, including for example sol-gel analysis determination of the gel fraction), swelling ratio analysis (determination of the swelling ratio), and mechanical analysis (e.g. determination of the modulus) (see e.g. Polym. Chem., 2024, 15, 219-247).

In an aspect, the fraction of the crosslinked polyester component (A) which is not soluble in ethyl acetate (gel fraction) is greater than or equal to 20%. In an aspect, the fraction of polyester elastomer which is not soluble in ethyl acetate (gel fraction) is greater than or equal to 40%. In an aspect, the fraction of the polyester elastomer which is not soluble in ethyl acetate (gel fraction) is greater than or equal to 50%. In an aspect, the fraction of the polyester elastomer which is not soluble in ethyl acetate (gel fraction) is greater than or equal to 60%. In an aspect, the fraction of the polyester elastomer which is not soluble in ethyl acetate (gel fraction) is greater than or equal to 70%. The gel fraction is suitably defined as

$$Gel fraction ( \% ) = 100 \times \frac{weight of dried gel ( insoluble residue of the extraction )}{total weight of the polyester elastomer used in the extraction} .$$

In an aspect, the gel fraction can be determined with an extraction method such as the Soxhlet extraction described herein.

In an aspect, crosslinked polyester component (A) is comprised of polyester elastomer and solvent or emollient as defined herein. In an aspect, polyester elastomer composition is comprised of polyester elastomer without solvent or emollient as defined herein. In an aspect, crosslinked polyester component (A) is a gel or a powder.

In an aspect, the crosslinked polyester component (A) is a polyester elastomer combined with one or more solvents or emollients, which can be converted to a polyester elastomer gel (the swollen polyester elastomer), for example, by applying a shear force to the composition.

### I. Definitions

Unless otherwise indicated, any atom with unsatisfied valences is assumed to have hydrogen atoms sufficient to satisfy the valences.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or” where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the following definitions shall apply unless otherwise indicated. For purposes of the present disclosure, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March's Advanced Organic Chemistry,” 6th Ed., Smith, M. B. and March, J., eds. John Wiley & Sons, New York: 2007, the entire contents of which are hereby incorporated by reference.

The term “hydrocarbon”, as used herein by itself or as part of a group, refers to a straight- or branched-chain aliphatic series of one to two hundred carbon atoms, i.e., a C1-C200 hydrocarbon, or the number of carbon atoms designated, e.g., a C1 hydrocarbon such as a methyl, a C2 hydrocarbon such as ethyl, etc. In one embodiment, the hydrocarbon is a C2-C200 hydrocarbon group. In an embodiment, the hydrocarbon is a C6-C60 hydrocarbon group. In an embodiment, the hydrocarbon is a C6-C60 hydrocarbon group. In an embodiment, the hydrocarbon is a C2-C60 hydrocarbon group. In an embodiment, the hydrocarbon is a C5-C22 hydrocarbon group. Examples of hydrocarbon groups include butyl, octyl, decyl, lauryl, cetyl (palmityl), and stearyl.

The term “alkyl”, as used herein by itself or as part of a group, refers to a straight or branched-chain aliphatic hydrocarbon containing one to two hundred carbon atoms, i.e., a C2-C200 alkyl, or the number of carbon atoms designated, e.g., a C1 alkyl such as methyl, a C2 alkyl such as ethyl, etc. In one embodiment, the alkyl is a C2-C200 alkyl group. In another embodiment, the alkyl is a C6-C60 alkyl group. In another embodiment, the alkyl is a C2-C60 alkyl group. In another embodiment, the alkyl is a C5-C22 alkyl group. Examples of alkyl group include butyl, octyl, decyl, lauryl, cetyl (palmityl), and stearyl.

The term “alkene”, as used herein by itself or as part of a group, refers to an alkyl group containing one, two, three, or more carbon-to-carbon double bonds. In one embodiment, the alkene group is a C2-C200 alkylene group. In another embodiment, the alkene group is a C6-C60 alkene group. In another embodiment, the alkene group is a C6-C60 alkene group. In another embodiment, the alkene group is a C2-C60 alkene group. In another embodiment, the alkene group is a C5-C22 alkene group.

The term “alkyne”, as used herein by itself or as part of a group, refers to an alkyl group containing one, two, three, or more carbon-to-carbon triple bonds. In another embodiment, the alkyne is a C2-C200 alkyne group.

The term “cyclic”, as used herein by itself or as part of a group, refers to a stable cyclic compound containing three or more atoms. In an embodiment, the cyclic is a C3-C200 cyclic group. In an embodiment, the cyclic is a C6-C60 cyclic group. In an embodiment, the cyclic is a C5-C22 cyclic group. Examples of cyclic compound include benzene, cyclopentane, and cyclohexane.

The term “heteroalkyl”, as used herein by itself or as part of a group, refers to a stable straight or branched chain alkyl radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkyl is a C6-C60 heteroalkyl group. In an embodiment, the heteroalkyl is a C2-C60 heteroalkyl group. Examples of heteroalkyl compound include succinyl, adipoyl, and sebacoyl.

The term “heteroalkene”, as used herein by itself or as part of a group, refers to a stable straight or branched chain alkene radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkene is a C6-C60 heteroalkene group. In an embodiment, the heteroalkene is a C2-C60 heteroalkene group. Examples of heteroalkene compound include oleoyl, ricinolyl, and linoleoyl.

The term “heteroalkyne”, as used herein by itself or as part of a group, refers to a stable straight or branched chain alkyne radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. The term “heterocyclic”, as used herein by itself or as part of a group, refers to a stable cyclic compound containing three or more atoms and at least one heteroatom, which can be the same or different, selected from O, N, or S. In an embodiment, the heterocyclic is a C2-C200 heterocyclic group. In an embodiment, the heterocyclic is C6-C60 heterocyclic group.

The term “heterocyclic”, as used herein by itself or as part of a group, refers to a stable cyclic compound containing two or more carbons atoms and at least one heteroatom, which can be the same or different, selected from O, N, or S, wherein the sulfur atom(s) can optionally be oxidized. In an embodiment, the heterocyclic is a C2-C200 heterocyclic group. In an embodiment, the heterocyclic is a C6-C60 heterocyclic group. In an embodiment, the heterocyclic is a C5-C22 heterocyclic group. Examples of heterocyclic compounds include furan, oxolane, and thiophene.

As used herein, the term “olefin” refers to any species having at least one ethylenic double bond such as normal and branched chain aliphatic olefins, cycloaliphatic olefins, aryl substituted olefins, and the like. An olefin can comprise terminal double bond(s) (“terminal olefin”) and/or internal double bond(s) (“internal olefin”) and can be cyclic or acyclic, linear or branched, optionally substituted. The total number of carbon atoms can be from 1 to 100, or from 1 to 40; the double bonds can be unsubstituted or mono-, bi-, tri- or tetrasubstituted.

As used herein, the term “polyolefin” refers to a homopolymer or copolymer of ethylene, propylene, butenes and other unsaturated aliphatic hydrocarbons, vinyl esters (e.g. vinyl acetate), or (meth)acrylics (e.g. butyl acrylate, acrylic acid). Generally, the polyolefin will be a polymer of ethylene, propylene or copolymer thereof, or a copolymer of ethylene or propylene with one or more C4-C12 α-olefin aliphatic comonomers.

A gel is a disperse system comprising at least two components: a solid component and a liquid component. The solid component forms a sponge-like, three-dimensional network whose pores are filled by a liquid. The liquid component is thus immobilized in the solid. In the gel of the invention the solid component is a three-dimensional network formed of the cross-linked polyester elastomer, and the liquid component is formed of one or more solvents or emollients as defined herein. A gel is a semi-solid that can have properties ranging from soft and weak to hard and tough. Gels are also defined as a substantially dilute cross-linked system.

An elastomer gel is thus made from the elastomer powders or particles swelled or dispersed in a liquid, such as a solvent or an emollient, to form a gel. The ability to swell is commonly expressed by the swelling ratio as explained herein.

The term “frizz” is defined as hair strands that are not aligned with surrounding hair, creating an irregular and often fuzzy texture. Frizz occurs more frequently with curly and wavy hair types.

The term “anti-frizz” ingredient refers to a component that either prevents hair frizz or reduces frizz by lubricating the hair and providing a boundary that encloses the hair cuticle. This prevents moisture from penetrating the hair and allows for a smoother hair feel and appearance. As will be shown below the crosslinked polyester component (A) surprisingly also provides an anti-frizz effect. In addition, further anti-frizz ingredients can be used as component (B) as will be explained below. Specific examples of additional anti-frizz ingredients are as follows: Lisselini by DKSH, Keranutri by Provital, Keratrix by Provital, Nori Complex by Provital, Andisil AS 220P by AB Specialty Silicones, SeraSense RB 21 by KCC Basildon, SeraSilk EL 60 by KCC Basildon (Momentive), Silsoft 1215 by Momentive Performance Materials, and DOW SIL CB-3046 fluid by DOW Chemical. Anti-frizz ingredients can also be natural or biobased, and include hydrolyzed keratin, argan oil, shea butter, coconut oil, glycerin, jojoba oil, macadamia nut oil, avocado oil, and cocoa butter. The list of these examples is not comprehensive.

The term “hair tress” is used to refer to a lock or curl of hair either separate from or growing out of a person's head.

The term “combing force” refers to a force experienced by a hair tress when a comb is pulled through the tress. When measuring the force used to comb a hair tress, a comb experiences resistance throughout the length of that tress. Combing ease is linked to friction and friction also plays a role in how consumers perceive hair conditions and feel.

The term “friction” is defined as the resistance that one surface encounters when moving over another surface. Evaluation of the friction of hair provides information about the condition of the hair, which can be affected by such things as the degree of cuticle lift, cuticle breakage, surface erosion and deposition of materials on the hair surface.

The term “coefficient of friction” is defined as a measure of the amount of friction existing between two surfaces. The reduction in coefficient of friction is significant in that hair exhibiting this characteristic has a greater ease of combing in the dry state.

The term “sensory panel” is defined as a group of individuals who have been trained to properly evaluate the different sensory attributes of personal care products, in this case, specifically hair care products. The attributes include, but are not limited to, slip (hair lengths and tips), coating, detangling, combing, curl definition, frizz/flyaway, reduction in volume, smoothness (hair lengths and tips).

The term “smoothing” refers to the effect of making hair softer, detangled and easier to handle, which is often achieved with a hair conditioning composition.

The term “softness” refers to the effect of making hair softer, shinier, and more supple, which is often achieved with a hair conditioning composition.

Ther term “detangling” refers to the process of either preventing tangles from forming or removing tangles from hair. This is especially significant in hair care to prevent and reduce hair cuticle damage and breakage.

The term “lubricating” refers to coating the hair cuticle, which increases slip and decreases friction between hair, and thus reduces tangles, frizz, damage, and the combing force required.

The term “curl defining” refers to enhancing existing waves and curls in one's hair by adding moisture, reducing frizz, and making curly and textured hair appear healthy and bouncy.

The term “conditioner base” refers to the chassis of hair conditioning compositions. These include, for example, cetearyl alcohol, cetyl alcohol, stearyl alcohol, glyceryl stearate, behentrimonium chloride, stearamidopropyl dimethylamine, cetrimonium chloride. Some of these conditioner bases also double as anti-frizz ingredients.

The term “medulla” refers the innermost layer of the hair shaft, which is the most delicate layer.

The term “cortex” is defined as the middle layer of the hair shaft, which is thickest and most responsible for the hair's width and pigment.

The term “cuticle” is defined as the outermost layer of the hair shaft, which consists of dead cells, and forms a barrier that protects the hair strand. The outer cuticle holds the hair shaft in the hair follicle by means of a bond. When it is healthy, it also minimizes the movement of water (moisture) in and out of the underlying cortex. However, chemical processes and weathering can lift the cuticle from the cortex and disrupt this balance. When the cuticle becomes damaged or broken, hair can appear frizzy, dry, lacking luster, or unhealthy.

The term “leave-in” products, in the context of hair care, refers to hair products such as conditioners that are left in hair after it has been washed in order to provide lasting moisture, protection, and de-tangling, among other benefits.

The term “rinse-off” products, in the context of hair care, refers to hair products such as shampoos, conditioners, serums, etc. that are washed out with water after they are applied to the hair.

The term “slip” refers to the slippery feeling of a product on hair, such as the lubrication of a conditioner or a leave-in product. Slip in hair is especially important to reduce friction and prevent damage and tangling.

The term “damage” when referring to hair refers most often to the damage of the outer layer of hair, or the hair cuticle, which can become cracked, stripped, or broken. This can occur due to heat, chemical treatments, color and dyes, and environmental damage.

The term “BTAC” refers to Behentrimonium chloride, which is a waxy, organic compound derived from the seeds of the Brassica rapa plant (canola or rapeseed oil). It is a conditioning agent that is commonly used in hair conditioners and hair masks.

The term “SAPDMA” refers to Stearamidopropyl Dimethylamine, a cationic surfactant and emulsifier commonly used in hair conditioners and other hair care products.

Cosmetically acceptable carriers (C) can be suitably selected from the group consisting of water, solvents, emollients, fatty acids, fatty alcohols, film formers, emulsifiers, thickeners and combinations thereof. Preferably they are selected from the solvents and emollients as described herein below. In particular the cosmetically acceptable carriers (C) can be also selected from the solvents and emollients described below, where these solvents and emollients are preferably used to provide component (A) as a gel in such solvents and emollients.

Various aspects of the disclosure are described in greater detail below.

### DESCRIPTION OF THE FIGURES

FIG. 1. The Effect of the Polyester Elastomer Component (A) on the Percent Improvement of Friction on Bleached European Hair Tresses

FIG. 2. The Effect of the Polyester Elastomer Component (A) on the Percent Improvement of Friction on Frizzy European Hair Tresses

FIG. 3. The Effect of the Polyester Elastomer Component (A) (Nat. Gel) on Percentage Change in Bulk Surface on Frizzy European Hair Tresses in BTAC Conditioner Base (Example 9)

FIG. 4. The Effect of the Polyester Elastomer Component (A) (Nat. Gel) on Percentage Change in Bulk Surface on Frizzy European Hair Tresses in SAPDMA Conditioner Base (Example 10)

FIG. 5. Photographs: Frizz Reduction Effect of the BTAC Conditioner Formulation of Example 9 (with 10% of Polyester Elastomer Component (A) (Natural Gel)) and Comparative Conditioner Formulation on Frizzy European Hair Tresses over 48 Hours

FIG. 6. Photographs: Frizz Reduction Effect of the SAPDMA Conditioner Formulation of Example 10 (with 10% of Polyester Elastomer Component (A) (Natural Gel)) and Comparative Conditioner Formulation on Frizzy European Hair Tresses over 48 Hours

### A. COMPONENTS OF THE CROSSLINKED POLYESTER COMPONENT (A)

### 1. Poly-Carboxylic Acids (i)

In some aspects, the at least one poly-carboxylic acid may be a compound of formula (VI)

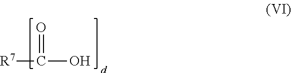

wherein
   R⁷ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
   d is an integer from 2 to 10.

In a further aspect, the compound is formula (I), wherein R¹ is C6-C60 alkyl group, C6-C60 heteroalkyl group, C6-C60 alkene group, C6-C60 heteroalkene group, C6-C60 cyclic group, or C6-C60 heterocyclic group; and a is an integer from 2 to 10. In an aspect, the compound is formula (I), wherein a is 2, 3, 4, 5, 6, 7, 8, 9, or 10. In an aspect, the compound is formula (I), wherein a is an integer from 2 to 6. In another aspect, the compound is formula (I), wherein a is 2, 3, 4, 5, or 6.

In an aspect, the polycarboxylic acid is a product of a compound of formula (I), and/or a compound of formula (III) with a compound of formula (V).

In an aspect, the at least one polycarboxylic acid may be selected from the group consisting of citric acid, isocitric acid, aconitic acid, propane-1,2,3-tricarboxylic acid, trimesic acid, carballylic acid, C54 trimer acid, mellitic acid, and combinations thereof. In a further aspect, the at least one polycarboxylic acid may be selected from the group consisting of citric acid, C54 trimer acid, and combinations thereof.

In an aspect, C36 dimer acid is a dicarboxylic acid prepared by dimerizing unsaturated linoleic fatty acid from plant oil.

In an aspect, C54 trimer acid is a polycarboxylic acid prepared by trimerizing unsaturated fatty acids from plant oil.

In some aspects, the unsaturated fatty acids are palmitoleic acid, oleic acid, elaidic acid, vaccenic acid, linoleic acid, linoelaidic acid, or linolenic acid.

In some aspects, the plant oils are soybean oil, safflower oil, linseed oil, corn oil, sunflower oil, olive oil, canola oil, sesame oil, cottonseed oil, palm oil, rapeseed oil, tung oil, peanut oil, or milkweed oil.

In an aspect, the at least one carboxylic acid may be a dicarboxylic acid. In an aspect, the dicarboxylic acid is a compound of formula (VII)

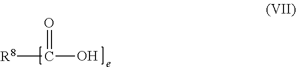

wherein
   R⁸ is C2-C200 alkyl group, C2-C200 heteroalkyl group, C2-C200 alkylene group, C2-C200 heteroalkylene group, C2-C200 alkyne group, C2-C200 heteroalkyne group, C3-C200 cyclic group, or C2-C200 heterocyclic group; and
   e is 2.

In another aspect, the compound is formula (VII), wherein R⁸ is C2-C200 alkyl group, C2-C200 heteroalkyl group, C2-C200 alkene group, or C2-C200 heteroalkene group and e is 2. In a further aspect, the compound is of formula (VII), wherein R⁸ is C2-C60 alkyl group, C2-C60 heteroalkyl group, C2-C60 alkene group, or C2-C60 heteroalkene group and e is 2. In an aspect, the compound is of formula (VII), wherein R⁸ may be succinyl, adipoyl, sebacoyl, dilinoleyl, or trilinoleyl.

In an aspect, the compound is formula (VII), wherein e may be 1 or 2.

In some aspects, the dicarboxylic acid may be selected from the group consisting of malonic acid, succinic acid, fumaric acid, glutaric acid, adipic acid, pimelic acid, suberic acid, azelaic acid, sebacic acid, undecanedioic acid, dodecanedioic acid, tridecanedioic acid, hexadecanedioic acid, C21 dimer acid, C36 dimer acid, hydrogenated C36 dimer acid, aspartic acid, glutamic acid, tartaric acid, malic acid, and combinations thereof. In a further aspect, the dicarboxylic acid may be selected from the group consisting of malonic acid, succinic acid, adipic acid, azelaic acid, sebacic acid, undecanedioic acid, dodecanedioic acid, C21 dimer acid, C36 dimer acid, hydrogenated C36 dimer acid, and combinations thereof. In one aspect, the dicarboxylic acid is selected from the group consisting of succinic acid, adipic acid, azelaic acid, sebacic acid, undecanedioic acid, dodecanedioic acid, C21 dimer acid, C36 dimer acid, hydrogenated C36 dimer acid, and combinations thereof.

In an aspect, the poly-carboxylic acid is biobased or naturally derived.

### 2. Polycarboxylic Acid Ester (i)

In an aspect, the at least one polycarboxylic acid ester may be a compound of formula (VIII)

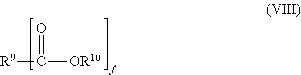

wherein
   R⁹ is C1-C22 alkyl group, C2-C22 alkylene group, or C3-C22 cyclic group;
   R¹⁰ is C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
   f is an integer from 2 to 10.

In another aspect, the compound is formula (VIII), wherein R⁹ is C1-C22 alkyl group or C2-C22 alkene group; R¹⁰ is C2-C200 alkyl group, C2-C200 heteroalkyl group, C2-C200 alkene group, or C2-C200 heteroalkene group; and f is an integer from 3 to 10.

In a further aspect, the compound is formula (VIII), wherein R⁹ is C1-C10 alkyl group; R¹⁰ is C2-C60 alkyl group, C2-C60 heteroalkyl group, C2-C60 alkene group, or C2-C60 heteroalkene group; and f is an integer from 3 to 10.

In an aspect, the compound is formula (VIII), wherein f is an integer from 3 to 6. In an aspect, the compound is formula (VIII), wherein f is 3, 4, 5, or 6.

In an aspect, the polycarboxylic acid ester is a product of a compound of formula (I), and/or a compound of formula (III) with a compound of formula (V).

In some aspects, the polycarboxylic acid ester may be selected from the group consisting of triethyl citrate, triethyl isocitrate, aconitic acid triethyl ester, propane-1,2,3-tricarboxylic acid triethyl ester, trimesic acid triethyl ester, carballylic acid triethyl ester, C54 trimer acid triethyl ester, mellitic acid hexaethyl ester, and combinations thereof. In a further aspect, the polycarboxylic acid ester may be selected from the group consisting of triethyl citrate, C54 trimer acid triethyl ester, and combinations thereof.

In an aspect, the at least one polycarboxylic acid ester may be a dicarboxylic acid ester. In some aspects, the dicarboxylic acid ester may be a compound of formula (IX)

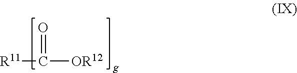

wherein
   R¹¹ is C1-C22 alkyl group, C2-C22 alkene group, or C3-C22 cyclic group;
   R¹² is C2-C200 alkyl group, C2-C200 heteroalkyl group, C2-C200 alkylene group, C2-C200 heteroalkene group, C2-C200 alkyne group, C2-C200 heteroalkyne group, C3-C200 cyclic group, or C2-C200 heterocyclic group; and
   g is 2.

In an aspect, the compound is formula (IX), wherein R¹¹ is C1-C22 alkyl group or C2-C22 alkylene group; R¹² is C2-C200 alkyl group, C2-C200 heteroalkyl group, C2-C200 alkene group, or C2-C200 heteroalkene group; and g is 2. In a further aspect, the compound is formula (IX), wherein R¹¹ is C1-C10 alkyl group; R¹² is C2-C60 alkyl group, C2-C60 heteroalkyl group, C2-C60 alkene group, or C2-C60 heteroalkene group; and g is 2.

In some aspects, the dicarboxylic acid may be selected from the group consisting of diethyl malonate, diethyl succinate, diethyl fumarate, diethyl adipate, diethyl pimelate, diethyl suberate, diethyl azelate, diethyl sebacate, diethyl undecanedioate, diethyl dodecanedioate, diethyl tridecanedioate, diethyl hexadecanedioiate, C21 dimer acid diethyl ester, C36 dimer acid diethyl ester, hydrogenated C36 dimer acid diethyl ester, diethyl aspartate, diethyl glutamate, diethyl tartrate, diethyl malate, and combinations thereof. In a further aspect, the dicarboxylic acid ester may be selected from the group consisting of diethyl malonate, diethyl succinate, diethyl adipate, diethyl pimelate, diethyl azelate, diethyl sebacate, diethyl undecanedioate, C21 dimer acid diethyl ester, C36 dimer acid diethyl ester, hydrogenated C36 dimer acid diethyl ester, and combinations thereof.

In an aspect, the poly-carboxylic acid ester is biobased or naturally derived.

### 3. Polyol (ii)

In an aspect, the at least one polyol is a compound of formula (X)

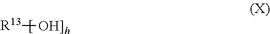

wherein
   R¹³ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
   h is an integer from 2 to 10.

In an aspect, the compound is formula (X), wherein R¹³ is C2-C200 alkyl group, C2-C200 heteroalkyl group, C2-C200 alkene group, or C2-C200 heteroalkene group; and h is an integer from 2 to 10. In a further aspect, the compound is formula (X), wherein R¹³ is C2-C60 alkyl group, C2-C60 heteroalkyl group, C2-C60 alkene group, or C2-C60 heteroalkene group; and h is an integer from 2 to 10.

In an aspect, the compound is formula (X), wherein h is an integer from 2 to 6. In an aspect, the compound is formula (X), wherein h is 2, 3, 4, 5, or 6.

In an aspect, the polyol is a product of a compound of formula (I), and/or a compound of formula (III) with a compound of formula (V).

In an aspect, the C36 dimer diol is the diol produced from a C36 dimer acid.

In some aspects, the polyol may be selected from the group consisting of glycerol, diglycerol, polyglycerol, sorbitan, castor oil, hydrogenated castor oil, sugar alcohol, monosaccharide, disaccharides, oligosaccharide, polysaccharides, tannin, gallic acid, gluconic acid, lactobionic acid, gluconolactone, and combinations thereof. In a further aspect, the polyol may be selected from the group consisting of glycerol, diglycerol, polyglycerol, castor oil, hydrogenated castor oil, sorbitol, gallic acid, and combinations thereof. In another aspect, the alcohol may be selected from the group consisting of glycerol, diglycerol, polyglycerol, castor oil, hydrogenated castor oil, sorbitol, and combinations thereof.

In some aspects, the at least one polyol is a diol. In some aspects, the diol is a compound of formula (XI)

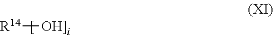

wherein
   R¹⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
   i is 2.

In an aspect, the compound is formula (XI), wherein R¹⁴ is C2-C200 alkyl group, C2-C200 heteroalkyl group, C2-C200 alkene group, or C2-C200 heteroalkene group; and i is 2. In a further aspect, the compound is formula (XI), wherein R¹⁴ is C2-C60 alkyl group, C2-C60 heteroalkyl group, C2-C60 alkene group, or C2-C60 heteroalkene group; and i is 2.

In some aspects, the diol may be selected from the group consisting of ethyleneglycol, 1,2-propanediol, 1,3-propanediol, 1,3-butanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C36 dimer diol, and combinations thereof. In a further aspect, the diol may be selected from the group consisting of 1,3-propanediol, 1,3-butanediol, 1,4-butanediol, 1,5-pentanediol, 1,6-hexanediol, C36 dimer diol, hydrogenated C36 dimer diol, and combinations thereof. In one aspect, the diol may be selected from the group consisting of 1,3-propanediol, 1,3-butanediol, 1,4-butanediol, 1,5-pentanediol, 1,6-hexanediol, C36 dimer diol, hydrogenated C36 dimer diol, and combinations thereof.

In an aspect, the polyol is biobased or naturally derived.

4. Mono-Carboxylic Acids (iii)(a)

In an aspect, the at least one mono-carboxylic acid (iii)(a) is a compound of formula (XII)

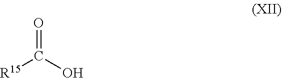

wherein
   R¹⁵ is C2-C52 alkyl group, C2-C52 heteroalkyl group, C2-C52 alkene group, C2-C52 heteroalkene group, C3-C52 cyclic group, or C2-C52 heterocyclic group.

In an aspect, the mono-carboxylic acid is a compound of formula (XII), wherein R¹⁵ is C5-C21 alkyl group, C5-C21 heteroalkyl group, C5-C21 alkene group, C5-C21 heteroalkene group, C5-C21 cyclic group, or C5-C21 heterocyclic group.

In an aspect, the mono-carboxylic acid is selected from the group consisting of caprylic acid, capric acid, lauric acid, myristic acid, palmitic acid, stearic acid, isostearic acid, behenic acid, oleic acid, linoleic acid, linolenic acid, and combinations thereof. In an aspect, the mono-carboxylic acid is oleic acid. In an aspect, the mono-carboxylic acid is isostearic acid.

In an aspect, R¹⁵ is C2-C52 alkyl group with at least one hydroxyl (—OH) group.

In an aspect, the mono-carboxylic acid is selected from the group consisting of hydroxylstearic acid, ricinoleic acid, isoricinoleic acid, lesquerolic acid, densipolic acid, auricolic acid, dimorphecolic acid, and combinations thereof. In an aspect, the mono-carboxylic acid is hydroxylstearic acid, ricinoleic acid, and combinations thereof.

In an aspect, the mono-carboxylic acid is biobased or naturally derived.

5. Mono-alcohols (iii)(b)

In an aspect, the at least one mono-alcohol is a compound of formula (XIII)

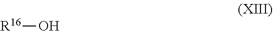

wherein
   R¹⁶ is C2-C52 alkyl group, C2-C52 heteroalkyl group, C2-C52 alkene group, C2-C52 heteroalkene group, C3-C52 cyclic group, or C2-C52 heterocyclic group.

In an aspect, the mono-alcohol is a compound of formula (XIII), wherein R¹⁶ is C5-C21 alkyl group, C5-C21 heteroalkyl group, C5-C21 alkene group, C5-C21 heteroalkene group, C5-C21 cyclic group, or C5-C21 heterocyclic group.

In an aspect, the mono-alcohol is selected from the group consisting of octanol, decanol, lauryl alcohol, myristyl alcohol, cetyl alcohol, stearyl alcohol, isostearyl alcohol, behenyl alcohol, oleyl alcohol, linoleyl alcohol, linolenyl alcohol, and combinations thereof. In an aspect, the mono-alcohol is oleyl alcohol. In an aspect, the mono-alcohol is isostearyl alcohol.

In an aspect, the mono-alcohol is biobased or naturally derived.

### B. RATIO OF COMPONENTS FOR THE PREPARATION OF CROSSLINKED POLYESTER COMPONENT (A)

In an aspect, the elastomer comprises a defined molar ratio of carboxyl functional groups (—COOH) from mono-carboxylic acid to hydroxyl functional groups (—OH) from polyol, and from hydroxyl functional groups (—OH) from mono-alcohol to carboxyl functional groups (—COOH) from poly-carboxylic acid. It has been found that the molar ratio of carboxyl functional groups (—COOH) from mono-carboxylic acid to hydroxyl functional groups (—OH) from the polyol and the molar ratio of hydroxyl functional groups (—OH) from mono-alcohol to carboxyl functional groups (—COOH) from poly-carboxylic acid influences the performance of the polyester elastomer and the performance of the gel made from the polyester elastomer.

In an aspect, the molar ratio of carboxyl functional groups (—COOH) from monocarboxylic acid to hydroxyl functional groups (—OH) from polyol is from about 1:2 to about 1:16. In an aspect, the molar ratio of carboxyl functional groups (—COOH) from mono-carboxylic acid to hydroxyl functional groups (—OH) from polyol is from about 1:2 to about 1:14. In an aspect, the molar ratio of carboxyl functional groups (—COOH) from mono-carboxylic acid to hydroxyl functional groups (—OH) from polyol is from about 1:2 to about 1:10. In an aspect, the molar ratio of carboxyl functional groups (—COOH) from mono-carboxylic acid to hydroxyl functional groups (—OH) from polyol is from about 1:2 to about 1:8. In an aspect, the molar ratio of carboxyl functional groups (—COOH) from mono-carboxylic acid to hydroxyl functional groups (—OH) from polyol is from about 1:2 to about 1:5. In an aspect, the molar ratio of carboxyl functional groups (—COOH) from mono-carboxylic acid to hydroxyl functional groups (—OH) from polyol is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of hydroxyl functional groups (—OH) from mono-alcohol to carboxyl functional groups (—COOH) from poly-carboxylic acid is from about 1:2 to about 1:16. In an aspect, the molar ratio of hydroxyl functional groups (—OH) from mono-alcohol to carboxyl functional groups (—COOH) from poly-carboxylic acid is from about 1:2 to about 1:14. In an aspect, the molar ratio of hydroxyl functional groups (—OH) from mono-alcohol to carboxyl functional groups (—COOH) from poly-carboxylic acid is from about 1:2 to about 1:10. In an aspect, the molar ratio of hydroxyl functional groups (—OH) from mono-alcohol to carboxyl functional groups (—COOH) from poly-carboxylic acid is from about 1:2 to about 1:8. In an aspect, the molar ratio of hydroxyl functional groups (—OH) from mono-alcohol to carboxyl functional groups (—COOH) from poly-carboxylic acid is from about 1:2 to about 1:5. In an aspect, the molar ratio of hydroxyl functional groups (—OH) from mono-alcohol to carboxyl functional groups (—COOH) from poly-carboxylic acid is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of carboxyl functional groups (—COOH) from poly-carboxylic acid to hydroxyl functional groups (—OH) from polyol is from about 1.5:1 to about 1:4. In an aspect, the ratio of carboxyl functional groups (~COOH) from poly-carboxylic acid to hydroxyl functional groups (—OH) from polyol is from about 1.5:1 to about 1:2. In an aspect, the ratio of carboxyl functional groups (—COOH) from poly-carboxylic acid to hydroxyl functional groups (—OH) from polyol is from about 1.5:1 to about 1:1.5. In an aspect, the ratio of carboxyl functional groups (—COOH) from poly-carboxylic acid to hydroxyl functional groups (—OH) from polyol is from about 1.5:1 to about 1:1.25. In an aspect, the ratio of carboxyl functional groups (—COOH) from poly-carboxylic acid to hydroxyl functional groups (—OH) from polyol is about 1.5:4, about 1.5:3, about 1.5:2, about 1:1, about 1:2, about 1:3, or about 1:4.

In an aspect, the conversion of carboxylic acid functional groups (—COOH) to ester functional groups (—CO(O)—) is no less than 80% by mole. The percent conversion is calculated by titration of carboxylic acid functional groups (—COOH) with 0.1 N KOH in isopropanol.

### C. METHODS OF PREPARING THE POLYESTER ELASTOMER COMPONENT (A)

### 1. Esterification Reaction

In one aspect, the present disclosure is directed to a method of preparing an elastomer (A) comprising reacting:
(i) at least one poly-carboxylic acid, at least one poly-carboxylic acid ester, or a combination thereof, and
(ii) at least one polyol; and
(iii) optionally one or more mono-carboxylic acids; and/or
(iv) optionally one or more mono-alcohols.

In an aspect, the elastomer prepared is a polyester elastomer (A). In an aspect, the elastomer prepared is a crosslinked polyester elastomer.

In an aspect, the preparation of the elastomer (A) is under nitrogen protection, is under vacuum, or is a combination thereof.

In an aspect, the elastomer is prepared by reacting:
(i) at least one poly-carboxylic acid, at least one poly-carboxylic acid ester, or a combination thereof, and
(ii) at least one polyol; and
(iii) at least one mono-carboxylic acid; and/or
(iv) at least one mono-alcohol.

In an aspect, the crosslinked polyester component (A) is reaction product of:
(i) one or more poly-carboxylic acids selected from di-carboxylic acids, tri-carboxylic acids and combinations thereof;
(ii) one or more polyols selected from diols, triols and combinations thereof, and
(iii) (a) optionally one or more monofunctional carboxylic acids.

In an aspect, the crosslinked polyester (A) is reaction product of:
(i) one or more dicarboxylic acids
(ii) one or more triols, and
(iii) (a) optionally one or more monofunctional carboxylic acids.

In an aspect, wherein the reaction comprised an activated di-carboxylic acid or tricarboxylic acid, the preparation of the elastomer further comprises addition of water to quench the activating agent from the reaction.

In an aspect, esterification is conducted in solvent or emollient. In another aspect, esterification is conducted in more than one solvent or emollient.

In an aspect, esterification is carried out in the absence of a solvent or emollient.

In an aspect the crosslinked polyester component (A) is obtained by a process comprising the steps of:
(a) reacting compounds (i), (ii) and optionally (iii) as defined in the claims,
   optionally in the presence of one or more solvents, to obtain a cross-linked polyester or a mixture thereof with the optional components,
b) optionally subjecting the crosslinked polyester or a mixture thereof with the optional components obtained in step (a) to a mechanical comminution process to obtain a cross-linked polyester component (A), or
c) optionally adding one or more solvents to the crosslinked polyester or a mixture thereof with the optional components obtained in step (a), allowing the resulting mixture to swell in said solvent, subjecting said swollen mixture to a mechanical comminution process and/or homogenization process in a reactor with planetary mixers to obtain a cross-linked polyester component (A) as a gel. In an aspect step (b) is carried out by mechanical stirring. In an aspect the mechanical comminution process step (c) is carried out with a three-roll mill.

### 2. Ratio of Components

In an aspect, the preparation of the elastomer comprises a defined ratio of poly-carboxylic acid, poly-carboxylic acid ester or a combination thereof (i) to polyol (ii), a defined ratio of monocarboxylic acid (iii)(a) to polyol (ii), and a defined ratio of mono-alcohol (iii)(b) to poly-carboxylic acid, poly-carboxylic acid ester, or a combination thereof (i). It has been found that the ratio of (i)/(iii)(a), (ii)/(iii)(a), and (iii)(b)/(i) influences the performance of the polyester elastomer and the performance of the gel made from the polyester elastomer.

In an aspect, the molar ratio of carboxyl functional groups (—COOH) from monocarboxylic acid (iii)(a) to hydroxyl functional groups (—OH) from polyol (ii) is from about 1:2 to about 1:16. In an aspect, the molar ratio of carboxyl functional groups (—COOH) from mono-carboxylic acid (iii)(a) to hydroxyl functional groups (—OH) from polyol (ii) is from about 1:2 to about 1:14. In an aspect, the molar ratio of carboxyl functional groups (—COOH) from mono-carboxylic acid (iii)(a) to hydroxyl functional groups (—OH) from polyol (ii) is from about 1:2 to about 1:10. In an aspect, the molar ratio of carboxyl functional groups (—COOH) from mono-carboxylic acid (iii)(a) to hydroxyl functional groups (—OH) from polyol (ii) is from about 1:2 to about 1:8. In an aspect, the molar ratio of carboxyl functional groups (—COOH) from mono-carboxylic acid (iii)(a) to hydroxyl functional groups (—OH) from polyol (ii) is from about 1:2 to about 1:5. In an aspect, the molar ratio of carboxyl functional groups (—COOH) from mono-carboxylic acid (iii)(a) to hydroxyl functional groups (—OH) from polyol (ii) is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of carboxyl functional groups (—COOH) from poly-carboxylic acid, poly-carboxylic acid ester or a combination thereof (i) to hydroxyl functional groups (—OH) from polyol (ii) is from about 1.5:1 to about 1:4. In an aspect, the ratio of carboxyl functional groups (—COOH) from poly-carboxylic acid, poly-carboxylic acid ester or a combination thereof (i) to hydroxyl functional groups (—OH) from polyol (ii) is from about 1.5:1 to about 1:2. In an aspect, the ratio of carboxyl functional groups (—COOH) poly-carboxylic acid, poly-carboxylic acid ester or a combination thereof (i) to hydroxyl functional groups (—OH) from polyol (ii) is from about 1.5:1 to about 1:1.5. In an aspect, the ratio of carboxyl functional groups (—COOH) poly-carboxylic acid, poly-carboxylic acid ester or a combination thereof (i) to hydroxyl functional groups (—OH) from polyol (ii) is from about 1.5:1 to about 1:1.25. In an aspect, the ratio of carboxyl functional groups (—COOH) poly-carboxylic acid, poly-carboxylic acid ester or a combination thereof (i) to hydroxyl functional groups (—OH) from polyol (ii) is about 1.5:4, about 1.5:3, about 1.5:2, about 1:1, about 1:2, about 1:3, or about 1:4.

In an aspect, the molar ratio of hydroxyl functional groups (—OH) from mono-alcohol (iii)(b) to carboxyl functional groups (—COOH) from poly-carboxylic acid (i) is from about 1:2 to about 1:16. In an aspect, the molar ratio of hydroxyl functional groups (—OH) from mono-alcohol (iii)(b) to carboxyl functional groups (—COOH) from poly-carboxylic acid (i) is from about 1:2 to about 1:14. In an aspect, the molar ratio of hydroxyl functional groups (—OH) from mono-alcohol (iii)(b) to carboxyl functional groups (—COOH) from poly-carboxylic acid (i) is from about 1:2 to about 1:10. In an aspect, the molar ratio of hydroxyl functional groups (—OH) from mono-alcohol (iii)(b) to carboxyl functional groups (—COOH) from poly-carboxylic acid (i) is from about 1:2 to about 1:8. In an aspect, the molar ratio of hydroxyl functional groups (—OH) from mono-alcohol (iii)(b) to carboxyl functional groups (—COOH) from poly-carboxylic acid (i) is from about 1:2 to about 1:5. In an aspect, the molar ratio of hydroxyl functional groups (—OH) from mono-alcohol (iii)(b) carboxyl functional groups (—COOH) from poly-carboxylic acid (A) is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the conversion of carboxyl functional groups (—COOH) to ester functional groups (—CO(O)—) is no less than 80% by mole. The percent conversion is calculated by titration of carboxylic acid functional groups (—COOH) with 0.1N KOH in isopropanol.

In an aspect, esterification is carried out in the absence of a solvent or emollient.

In an aspect, esterification is conducted in solvent or emollient. In another aspect, esterification is conducted in more than one solvent or emollient.

In an aspect, the percentage of solvent or emollient in total raw materials of the esterification reaction is in the range of 0% to 80% by weight. In an aspect, the percentage of solvent or emollient in total raw materials of the esterification reaction is in the range of 0% to 60% by weight. In an aspect, the percentage of solvent or emollient in total raw materials of the esterification reaction is in the range of 0% to 50% by weight. In an aspect, the percentage of solvent or emollient in total raw materials of the esterification reaction is in the range of 0% to 40% by weight. In an aspect, the percentage of solvent or emollient in total raw materials of the esterification reaction is in the range of 0% to 30% by weight. In an aspect, the percentage of solvent or emollient in total raw materials of the esterification is in the range of 0% to 20% by weight. In an aspect, the percentage of solvent or emollient in total raw materials of the esterification reaction is in the range of 0% to 10% by weight. In an aspect, the percentage of solvent or emollient in total raw materials of the esterification reaction is in the range of 10% to 20% by weight. In an aspect, the percentage of solvent or emollient in total raw materials of the esterification reaction is in the range of 20% to 30% by weight.

### 3. Activating Agent

In an aspect, the preparation of the elastomer further comprises an activating agent. In an aspect, the preparation of the elastomer does not comprise an activating agent.

In an aspect, the activating agent is selected from the group consisting of dimethyl dicarbonate, diethyl dicarbonate, dipropyl dicarbonate, di-tertiary-butyl dicarbonate, and combinations thereof.

### 4. Catalyst

In an aspect, the preparation of the elastomer further comprises a catalyst. In an aspect, the preparation of the elastomer does not comprise a catalyst. However, it has been found that when no catalyst is used the reaction times are protracted.

In an aspect, the catalyst is selected from the group consisting of methanesulfonic acid, p-toluenesulfonic acid, benzene sulfonic acid, sulfuric acid, amidosulfonic acid, sulfamic acid, sodium bisulfate, phosphoric acid, hydrochloric acid, hydrobromic acid, nitric acid, sodium hydroxide, potassium hydroxide, sodium methoxide, sodium ethoxide, potassium methoxide, potassium ethoxide, bismuth(III) neodecanoate, bismuth(III) citrate, bismuth(III) chloride, bismuth(III) acetate, bismuth(III) phosphate, tin chloride, tin-pyrone, dibutyltin dilaurate, di-n butyl-oxo-stannane, butyl stannoic acid, zinc chloride, zinc bromide, zinc carboxylic salt, zinc oxide, zinc hydroxy nitrate salt, zinc hydroxy acetate, triethylamine, tripropylamine, cocamidopropyl dimethylamine, stearamidopropyl dimethylamine, isostearamidopropyl dimethylamine, and combinations thereof. In an aspect, the catalyst is p-toluenesulfonic acid, methanesulfonic acid, phosphoric acid, bismuth neodecanoate, or a combination thereof. In an aspect, the catalyst is methanesulfonic acid.

In an aspect, the catalyst is a salt. In an aspect, the catalyst is a salt selected from the group consisting of Yb(OTf)₃, Sc(OTf)₃, Hf(OTf)₄, Bi(OTf)₃, Al(OTf)₃, Zn(OTf)₂, Mg(ClO₄)₂, Cu(OTf)₂, Ti(OCH(CH₃)₂)₄, and combinations thereof.

### 5. Emollients or Solvents

In an aspect the cosmetically acceptable carrier (C) is preferably selected from the emollients or solvents as described herein below. The cosmetically acceptable carrier (C) can be present in the preparation of the crosslinked polyester component (A) and/or can be added after the preparation of the crosslinked polyester component (A).

In an aspect, the preparation of the polyester elastomer (A) can occur in the presence of a solvent. The solvent can also act as an emollient, preferably a cosmetic emollient. When the solvent acts also as an emollient it may also provide a softening, protecting, moisturizing, and/or lubricating effect to the hair and skin. In an aspect, the solvent or emollient is preferably a biobased or naturally derived. In an aspect, the solvent or emollient is a triglyceride solvent, a mono-ester solvent, a di-ester solvent, a citrate ester solvent, an ether solvent, a carbonate solvent, a hydrocarbon solvent, a silicone solvent, or a combination thereof.

In an aspect, the solvent is a triglyceride solvent of formula (XIV)

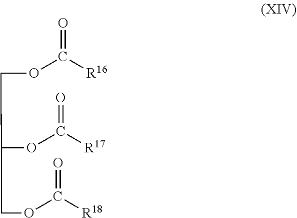

wherein
   each R¹⁶, R¹⁷, and R⁸ are independently C1-C35 alkyl group, C1-C35 heteroalkyl group, C2-C35 alkene group, or C2-C35 heteroalkene group.

In an aspect, the solvent is of formula (XIV), wherein R¹⁶, R¹⁷, and R¹⁸ are independently C2-C17 alkyl group or C2-C17 alkylene group.

In an aspect, the solvent is a triglyceride solvent selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof. In an aspect, the triglyceride solvent is selected from the group consisting of caprylic/capric triglyceride, triheptanoin, and combinations thereof.

In an aspect, the solvent is a mono-ester solvent of formula (XV)

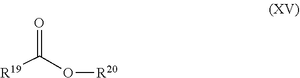

wherein
   R¹⁹ is C1-C35 alkyl group, C1-C35 heteroalkyl group, C2-C35 alkene group, or C2-C35 heteroalkene group; and
   R²⁰ is C1-C35 alkyl group, C1-C35 heteroalkyl group, C2-C35 alkene group, or C2-C35 heteroalkene group.

In an aspect, the solvent is a mono-ester solvent of formula (XV), wherein R¹⁹ is C5-C17 alkyl group or C5-C17 alkene group and R²⁰ is C2-C17 alkyl group or C2-C17 alkene group.

In an aspect, the solvent is a mono-ester solvent selected from the group consisting of coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octyldodecyl erucate, C12-C13 alkyl lactate, C12-C15 alkyl lactate, isostearyl lactate, glycereth-5-lactate, lauryl lactate, myristyl lactate, oleyl lactate, laureth-2-benzoate, C12-C15 alkyl benzoate, C12-C15 pareth-3-benzoate, dipropylene glycol benzoate, isodecyl salicylate, C12-C15 alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2-ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof. In an aspect, the mono-ester solvent is selected from the group consisting of cococaprylate/caprate, coco-caprylate, jojoba oil, isoamyl laurate, methylheptyl isostearate, C12-C13 alkyl lactate, C12-C15 alkyl lactate, lauryl lactate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, and combinations thereof. In an aspect, the mono-ester solvent is selected from the group consisting of coco-caprylate/caprate, coco-caprylate, isoamyl laurate, isononyl isononanoate, heptyl undecylenate, jojoba oil, jojoba esters, and combinations thereof.

In an aspect, the solvent is:
(a) a di-ester solvent of formula (XVI)

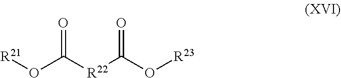

wherein
   R²¹ is C1-C35 alkyl group, C1-C35 heteroalkyl group, C2-C35 alkene group, or C2-C35 heteroalkene group; and
   R²² and R²³ are independently C1-C35 alkyl group, C1-C35 heteroalkyl group, C2-C35 alkene group, or C2-C35 heteroalkene group; or
(b) a di-ester solvent of formula (XVII)

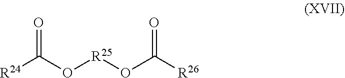

wherein
   R²⁴ is C1-C35 alkyl group, C1-C35 heteroalkyl group, C2-C35 alkene group, or C2-C35 heteroalkene group; and
   R²⁵ and R²⁶ are independently H, C1-C35 alkyl group, C1-C35 heteroalkyl group, C2-C35 alkene group, or C2-C35 heteroalkene group; or
(c) a di-ester solvent of formula (XVIII)

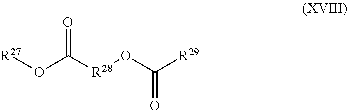

wherein
   R²⁷ and R²⁸ are independently C1-C35 alkyl group, C1-C35 heteroalkyl group, C2-C35 alkene group, or C2-C35 heteroalkene group; and
   R²⁹ is H, C1-C35 alkyl group, C1-C35 heteroalkyl group, C2-C35 alkene group, or C2-C35 heteroalkene group.

In an aspect, the solvent is a di-ester solvent of formula (XVI), formula (XVII), or formula (XVIII), wherein R²³, R²⁶, and R²⁹ is C2-C10 alkyl group or C2-C10 alkene group and R²¹, R²⁴, R²⁷, and R²², R²⁵, and R²⁸ are independently C1-C12 alkyl group or C2-C12 alkene group.

In an aspect, the di-ester solvent is selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof. In an aspect, the di-ester solvent is selected from the group consisting of dicapryl adipate, dicaprylyl maleate, diisopropyl adipate, diisobutyl adipate, diethyl succinate, dibutyl succinate, diethyhexyl succinate, diisopropyl sebacate, dimethyl sebacate, diethyl sebacate, dibutyl sebacate, neopentyl glycol diethylhexanoate, neopentyl glycol diheptanoate, and combinations thereof.

In an aspect, the solvent is a citrate ester solvent of formula (XVIII)

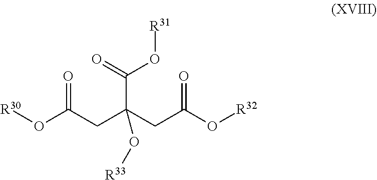

wherein
   R³⁰, R³¹, R³², and R³³ are independently H, C1-C35 alkyl group, C1-C35 heteroalkyl group, C2-C35 alkene group, or C2-C35 heteroalkene group, wherein at least one of R³⁰, R³¹, R³², and R³³ is not H.

In an aspect, the solvent is a citrate ester solvent of formula (XVIII), wherein R³⁰, R³¹, and R³² are independently C1-C10 alkyl group or C2-C10 alkene group and R³³ is an acetyl group.

In an aspect, the solvent is a citrate ester solvent selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof.

In an aspect, the solvent is an ether solvent of formula (XIX)

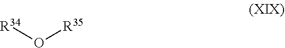

wherein
   R³⁴ and R³⁵ are independently H, C2-C20 alkyl group, C2-C20 heteroalkyl group, C2-C20 alkene group, or C2-C20 heteroalkene group, wherein at least one of R³⁴ and R³⁵ is not H.

In an aspect, the solvent is an ether solvent of formula (XIX), wherein R³⁴ and R³⁵ are independently C2-C20 alkyl group.

In an aspect, the solvent is an ether solvent selected from the group consisting of dicaprylyl ether, didecyl ether, panthenyl ethyl ether, dicetyl ether, dimyristyl ether, distearyl ether, dilauryl ether, and combinations thereof. In an aspect, the ether solvent is selected from the group consisting of dicaprylyl ether, didecyl ether, and combinations thereof.

In an aspect, the solvent is a carbonate solvent of formula (XX)

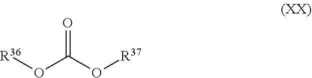

wherein
   R³⁶ and R³⁷ are independently H, C2-C20 alkyl group, C2-C20 heteroalkyl group, C2-C20 alkene group, or C2-C20 heteroalkene group.

In an aspect, the solvent is a carbonate solvent of formula (XX), wherein R³⁶ and R³⁷ are independently C2-C20 alkyl group.

In an aspect, the solvent is a carbonate solvent selected from the group consisting of dicaprylyl carbonate, diethyl hexyl carbonate, and combinations thereof.

In an aspect, the solvent is a hydrocarbon with number of carbon atoms from C4 to C60. In an aspect, the solvent is a hydrocarbon with a number of carbon atoms from C10 to C50. In an aspect, the solvent is a hydrocarbon with a number of carbon atoms from C20 to C40.

In an aspect, the solvent is a hydrocarbon solvent selected from the group consisting of farnesene, hydrogenated farnesene, coconut alkanes, coconut/palm kernel alkanes, C9-C12 alkane, C10-C13 alkane, C12-C17 alkane, C13-C14 alkane, C13-C15 alkane, C14-C17 alkane, C14-C19 alkane, C14-C20 alkane, C14-C22 alkane, C15-C19 alkane, C21-C28 alkane, C17-C23 alkane, C9-C12 isoalkane, C9-C13 isoalkane, C9-C14 isoalkane, C9-C16 isoalkane, C10-C11 isoalkane, C10-C12 isoalkane, C10-C13 isoalkane, C11-C12 isoalkane, C11-C13 isoalkane, C11-C14 isoalkane, C12-C14 isoalkane, C12-C15 isoalkane, C12-C20 isoalkane, C13-C14 isoalkane, C13-C16 isoalkane, C14-C16 isoalkane, C15-C19 isoalkane, C10-C16 olefin, C12-C18 olefin, C18-C26 olefin, C20 olefin, C20-C24 olefin, C24-C30 olefin, C26-C28 olefin, C26-C54 olefin, C28-C36 olefin, C28-C52 olefin, C30-C38 olefin, C30-C45 olefin, C4-C12 olefin, C4-C6 olefin, C5-C6 olefin, hydrogenated poly(C6/C10/C14 olefin), hydrogenated poly(C6-C12 olefin), hydrogenated poly(C6-C14 olefin), hydrogenated poly(C6-C20 olefin), hydrogenated poly(C8/C12 olefin), poly(C20-C28 olefin), poly(C30-C45 olefin), poly(C4-C12 olefin), poly(C6-C14 olefin), hexadecene, C32 alkane, C32 isoalkane, C54 alkane, C54 isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof. In an aspect, the hydrocarbon solvent is selected from the group consisting of squalane, farnesene, hydrogenated farnesene, coconut alkanes, C9-C12 alkane, C13-C15 alkane, C14-C19 alkane, C14-C20 alkane, C14-C22 alkane, C15-C19 alkane, C13-C16 isoalkane, dodecane, undecane, tridecane, tetradecane, pentadecane, hexadecane, hexadecene, octadecane, squalane, isododecane, isohexadecane, C32 alkane, C32 isoalkane, C54 alkane, C54 isoalkane, and combinations thereof. In an aspect, the hydrocarbon solvent is selected from the group consisting of squalane, hydrogenated farnesene, coconut alkanes, C9-C12 alkane, C13-C15 alkane, C13-C16 isoalkane, C14-C19 alkane, dodecane, tetradecane, isododecane, hexadecane, octadecane, hexadecene, C32 alkane, C32 isoalkane, C54 alkane, C54 isoalkane, and combinations thereof.

In an aspect, the hydrocarbon solvent is selected from the group consisting of squalane, C32 alkane, C32 isoalkane, C54 alkane, C54 isoalkane, and combinations thereof.

In an aspect, the solvent may be also a chemical UV filter. The UV filter solvent may be selected from the group consisting of ethyl dimethyl PABA, ethylhexyl methoxycinnamate, ethylhexyl salicylate, homosalate, isoamyl p-Methoxycinnamate, menthyl anthranilate, octocrylene, polysilicone-15, terephthalyidene dicamphor sulfonic acid, triethanolamine salicylate, and combinations thereof.

In an aspect, the solvent is a silicone solvent selected from the group consisting of dimethicone, phenyl dimethicone, caprylyl methicone, ethyl trisiloxane, cyclotetrasiloxane, cyclopentasiloxane, cyclohexasiloxane, and combinations thereof.

In an aspect, the solvent or emollient is preferably a triglyceride solvent, a mono-ester solvent, a di-ester solvent, a citrate ester solvent, an ether solvent, a carbonate solvent, a hydrocarbon solvent, a silicone solvent, or a combination thereof, each as described more specifically before.

In an aspect, a defined amount of solvent is used in the preparation of the polyester elastomer. In an aspect, the amount of solvent is from 0% to 70% of the total weight of poly-carboxylic acid (i), polyol (ii), mono-carboxylic acid (iii)(a) and mono-alcohol (iii)(b). In an aspect, the amount of solvent is from 0% to 50% of the total weight of poly-carboxylic acid (i), polyol (ii), mono-carboxylic acid (iii)(a) and mono-alcohol (iii)(b). In an aspect, the amount of solvent is from 0% to 40% of the total weight of poly-carboxylic acid (i), polyol (ii), mono-carboxylic acid (iii)(a) and mono-alcohol (iii)(b). In an aspect, the amount of solvent is from 0% to 30% of the total weight poly-carboxylic acid (i), polyol (ii), mono-carboxylic acid (iii)(a) and mono-alcohol (iii)(b). In an aspect, the range of solvent is from 0% to 20% of the total weight of poly-carboxylic acid (i), polyol (ii), mono-carboxylic acid (iii)(a) and mono-alcohol (iii)(b). In an aspect, the amount of solvent is from 10% to 50% of the total weight of poly-carboxylic acid (i), polyol (ii), mono-carboxylic acid (iii)(a) and mono-alcohol (iii)(b). In an aspect, the amount solvent is 50%, 40%, 30%, 20%, or 10% of the total weight of poly-carboxylic acid (i), polyol (ii), mono-carboxylic acid (iii)(a) and mono-alcohol (iii)(b).

In an aspect, the amount of solvent is from 0% to 30% of the total weight of poly-carboxylic acid (i) and mono-carboxylic acid (iii)(a). In an aspect, the amount of solvent is from 0% to 20% of the total weight of poly-carboxylic acid (i) and mono-carboxylic acid (iii)(a). In an aspect, the amount of solvent is from 0% to 10% of the total weight of poly-carboxylic acid (i) and mono-carboxylic acid (iii)(a). In an aspect, the amount of solvent is from 10% to 30% of the total weight of poly-carboxylic acid (i) and mono-carboxylic acid (iii)(a). In an aspect, the solvent is 30%, 20%, or 10% of the total weight of poly-carboxylic acid (i) and mono-carboxylic acid (iii)(a).

In an aspect, no solvent is used in the preparation of the polyester elastomer (A).

In an aspect, a solvent is used to prepare the polyester elastomer (A), which is removed after preparing the polyester elastomer (A) to form a polyester elastomer (A) powder or which is left in the polyester elastomer (A) to provide a gel optionally after a shearing step. In an aspect, to the polyester elastomer powder a solvent/emollient can be added after the manufacture of the polyester elastomer (A) to form a polyester elastomer (A) gel, optionally applying a shear force as described below.

In an aspect, polyester elastomer (A) is made from C36 dimer acid, diglycerol, and isostearic acid with about 10% to about 40% by weight squalane based on the total weight of the polyester elastomer (A) and the squalane. That is, in an aspect the invention relates to a composition of a polyester elastomer made from C36 dimer acid, diglycerol, isostearic acid, and squalane, comprising about 10% to about 40% by weight squalane.

In an aspect, polyester elastomer (A) is made from hydrogenated C36 dimer acid, diglycerol, and oleic acid without any solvent or emollient.

### 6. Temperature

In an aspect, the method of preparing the elastomer comprises reacting at least one poly-carboxylic acid, poly-carboxylic acid ester, or combination thereof, at least one polyol, and/or at least one mono-carboxylic acid, and/or at least one mono-alcohol with mixing at a pre-determined temperature until an elastomer is formed. In an aspect, the method of preparing the elastomer comprises reacting at least one poly-carboxylic acid, poly-carboxylic acid ester, or combination thereof, at least one polyol, and/or at least one mono-carboxylic acid, and/or at least one mono-alcohol, optionally at least one solvent or emollient, and optionally a catalyst with mixing at a pre-determined temperature until an elastomer is formed. In an aspect, the temperature range is from 30° C. to 250° C.

In an aspect, the reaction occurs at a temperature from about 30° C. to about 250° C. In an aspect, the reaction occurs at a temperature from about 60° C. to about 250° C. In an aspect, the reaction occurs at a temperature from about 30° C. to about 125° C. or about 40° C. to about 100° C. In an aspect, the reaction occurs at a temperature of about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., about 200° C., about 205° C., about 210° C., about 215° C., about 220° C., about 225° C., about 230° C., about 235° C., about 240° C., about 245° C., or about 250° C.

### 7. Time

In an aspect, the reaction time is from about 12 hours to about 150 hours. In an aspect, the reaction time is from about 6 hours to about 24 hours. In an aspect, the reaction time is from about 8 hours to about 27 hours. In an aspect, the reaction time is about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5 hours, about 26 hours, about 26.5 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 55 hours, about 60 hours, about 65 hours, about 70 hours, about 75 hours, about 80 hours, about 85 hours, about 90 hours, about 95 hours, about 100 hours, about 105 hours, about 110 hours, about 115 hours, about 120 hours, about 125 hours, about 130 hours, about 135 hours, about 140 hours, about 145 hours, or about 150 hours.

The reaction time can be adjusted by determination of the gel fraction achieved, and preferably the reaction time is such that the gel fraction of the polyester elastomer is greater than 60%. A method to measure the gel fraction in the polyester elastomer is described below.

### 8. By-Product Removal

In an aspect, the method further comprises removing water and alcohol by-product from the reaction. In a further aspect, the water and alcohol by-products are removed from the reaction by mixing and heating the reaction. In an aspect, the reaction is heated to above about 120° C. to remove the water and alcohol by-products. In an aspect, the water and alcohol by-products are removed from the reaction by nitrogen flow, by vacuum, or a combination thereof. In an aspect, water is removed by nitrogen stripping and vacuum, which have an impact on the reaction time.

As mentioned above in an aspect, the polyester elastomer (A) is provided as a (at room temperature (23° C.) flowable or fluid) gel or a (dry) powder.

In an aspect, the polyester elastomer (A) is processed into a gel as described herein.

In an aspect, polyester elastomer (A) used is only comprised of crosslinked polyester without solvent or emollient. In an aspect, polyester elastomer (A) used is comprised of polyester without solvent or emollient. In an aspect, polyester elastomer (A) used is comprised of crosslinked polyester with solvent or emollient. In an aspect, polyester elastomer (A) used is comprised of polyester with solvent or emollient.

Solvents or emollients that can be used to prepare the polyester elastomer composition (A) are as described herein above and can be selected from the solvents or emollients as defined herein. In another aspect, the polyester elastomer in polyester elastomer composition (A) used is in the range of 5 wt % to 100 wt %. In another aspect, the polyester elastomer in polyester elastomer composition (A) used is in the range of 5 wt % to 70 wt %. In another aspect, the polyester elastomer in polyester elastomer composition (A) used is in the range of 10 wt % to 60 wt %. In another aspect, the polyester elastomer in polyester elastomer composition (A) used is in the range of 20 wt % to 50 wt %.

In another aspect, the crosslinked polyester in polyester elastomer composition (A) used is in the range of 5 wt % to 50 wt %. In another aspect, the crosslinked polyester in polyester elastomer composition (A) used is in the range of 5 wt % to 30 wt %. In another aspect, the crosslinked polyester in polyester elastomer composition (A) used is in the range of 10 wt % to 30 wt %. Solvents or emollients that can be used to prepare the polyester elastomer composition are described herein and can be selected from the solvents or emollients as defined herein.

In an aspect, the polyester elastomer composition (A) used comprises at least one solvent or emollient added to the polyester elastomer during the shearing force process to form a gel. Solvents or emollients that can be used to prepare the polyester elastomer composition are described herein and can be selected from the solvents as defined herein. In an aspect, the solvent or emollient is from about 20% to about 95% weight by weight of the composition. In an aspect, the solvent or emollient is from about 20% to about 50% weight by weight of the composition. In an aspect, the solvent or emollient is from about 50% to about 90% weight by weight of the composition. In an aspect, the solvent or emollient is from about 70% to about 90% weight by weight of the composition. In an aspect, the polyester elastomer composition (A) comprises from about 50% to about 90% weight by weight of solvent or emollient, from about 50% to about 80% weight by weight of solvent or emollient, from about 50% to about 70% weight by weight of solvent or emollient, or from about 50% to about 60% weight by weight of solvent or emollient. In some embodiments, the polyester elastomer composition (A) comprises about 20%, about 30%, about 40%, about 50%, about 60%, about 70%, about 80%, or about 90% weight by weight of solvent or emollient.

### Process to Prepare the Polyester Elastomer Component (A) in the Form of a Gel Composition

In an aspect, the polyester elastomer obtained by the esterification process is crumbled or processed to form a polyester elastomer powder. In another aspect, the polyester elastomer is processed by a three-roll mill to form a polyester elastomer powder.

The polyester elastomer composition (A) can be also obtained as a powder if it still comprises a solvent or emollient added to the reaction mixture.

In an aspect, polyester elastomer composition (A) is prepared by combining the polyester elastomer with one or more solvents or emollients to form a polyester elastomer gel.

In an aspect, polyester elastomer and solvent/emollient mixture are processed with a homogenizer to produce a gel optionally applying a shear force, e.g. by a high-shear disperser mixer.

In an aspect to prepare the polyester elastomer composition (A) as gel, a polyester elastomer is swelled in a solvent or emollient before being processed to make a gel at a temperature under 23° C. In an aspect, the time of polyester elastomer swelling in a solvent or emollient is from 1 hour to 1 week. In an aspect, the elastomer is subject to swelling in a solvent or emollient from 10 minutes to 1 week, from 10 minutes to 4 days, from 10 minutes to 3 days, from 10 minutes to 2 days, from 10 minutes to 1 day, from 10 minutes to 12 hours, from 10 minutes to 6 hours, from 10 minutes to 3 hours, from 10 minutes to 2 hours, from 10 minutes to 1 hour, or from 10 minutes to 30 minutes.

Once the initially produced polyester elastomer is prepared, it can be mixed with an additional quantity of at least one solvent or emollient that can be different from the solvent emollient used to prepare the initially produced elastomer. In some aspects, the at least one solvent or emollient used to prepare the elastomer is the same as the at least one solvent or emollient used to prepare the elastomer composition. The addition of an additional quantity of at least one solvent or emollient dilutes the gel composition and thereby adjusts its viscosity.

The method of preparing the polyester elastomer composition (A) as gel, suitably comprises:
(i) combining a polyester elastomer with at least one solvent or emollient thereby forming a swollen polymer elastomer; and
(ii) subjecting the swollen polyester elastomer to shear force thereby forming a polyester elastomer composition.

In an aspect, the polyester elastomer composition is provided as a powder, a gel, or a paste.

In an aspect, the at least one solvent or emollient is selected from the solvents or emollients described herein.

### Characterization of the Crosslinked Polyester Components (A)

In an aspect, the solubility of polyester elastomer (A) is measured by mixing 1 gram of polyester elastomer with 100 gram of test solvent with magnetic stirring in a sealed glass container for 24 hours under room temperature (23° C.). Afterward the polyester elastomer (A) and test solvent is passed through filtration and the solid on filter is obtained and dried under 80° C. for 20 hours or dried to constant weight, optionally in vacuo. The dried solid is considered as the fraction of polyester elastomer (A) which is not soluble in the test solvent.

In an aspect, the fraction of the polyester elastomer (A) which is not soluble in the ethyl acetate by weight is greater than or equal to 40% of the total weight of the polyester elastomer (A).

In another aspect, the fraction of the polyester elastomer (A) which is not soluble in ethyl acetate is 40% to 90% of the total weight of the polyester elastomer (A). In another aspect, the fraction of the polyester elastomer (A) which is not soluble in ethyl acetate is 50% to 90% of the total weight of the polyester elastomer (A).

A sample of polyester elastomer (A) without solvent or emollient, or a sample of polyester elastomer (A) with solvent or emollient can be used. In case a sample of polyester elastomer (A) with solvent or emollient is used, the weight of the solvent or emollient in the composition is considered and subtracted from the total composition weight to determine the weight percentage of the fraction of the polyester elastomer (A) which is not soluble in the ethyl acetate.

The method of Soxhlet extraction can be used to determine the % by weight of crosslinked polyester (gel fraction) in the polyester elastomer (A). The basis for the test is to extract soluble low Mw components (and optionally present solvents or emollients) from the high Mw and crosslinked insoluble components through an extraction test. The percentage of the crosslinked polyester is defined as the ratio of the weight of insoluble residue (dried gel) to the initial weight of the polyester elastomer sample.

The gel fraction of the polyester elastomers (A), defined as

$$Gel fraction ( \% ) = 100 \times \frac{weight of dried gel ( insoluble residue of the extraction )}{total weiht of the polyester elastomer used in the extraction}$$

is determined as follows.

A sample of polyester elastomer without solvent or emollient, or a sample of polyester elastomer with solvent or emollient can be used. In case a sample of polyester elastomer with solvent or emollient is used, the weight of the solvent or emollient in the polyester elastomer is considered and subtracted from the sample weight to determine the total weight of the polyester elastomer (A) used in the extraction in the formula above.

A cellulose extraction thimble is weighed. 3.0-3.5 grams of a sample of a polyester elastomer is weighed and placed in the cellulose extraction thimble. About 125-150 mL of ethyl acetate (EtOAc) are placed in a 250 mL round bottom flask. The thimble with the sample is placed into the Soxhlet extraction column. The EtOAc solution is heated to 80° C. (Reflux, bp 77° C.) and maintained at reflux for 1 hour to extract the polymer sample solubles and the optional present solvent or emollient. After 2 hours the EtOAc solution is allowed to cool to room temperature (about 23° C.). The thimble containing the polymer residue is removed from the Soxhlet apparatus. The thimble is placed in a desiccator under vacuum, a vacuum oven, or a vented oven (50° C.) to remove the residual EtOAc (24 hours). After the EtOAc has been removed, the weight of the thimble containing the dried gel is determined. The weight of the dried gel is calculated by subtraction of the weight of the cellulose extraction thimble. The gel fraction is calculated from the above equation. Any amount of solvent or emollient in the sample is subtracted from the “total weight of the polyester elastomer (A) used in the extraction”.

In an aspect, the gel fraction of the polyester elastomer (A) is greater than 20%. In an aspect, the gel fraction of the polyester elastomer (A) is greater than 40%. In an aspect, the gel fraction of the polyester elastomer (A) is greater than 50%. In an aspect, the gel fraction of the polyester elastomer is greater than 60%. In an aspect, the gel fraction of the polyester elastomer (A) is greater than 70%.

Swelling test for the polyester elastomer (A) is to determine the swelling capacity of the polyester elastomer (A) through a weight of solvent or emollient retained by the polyester elastomer. The swelling ratio (SR—sometimes called swelling value) is determined according to the equation:

$$SR = \frac{Ws - Wi}{Wi}$$

where:
   Ws is the weight of the swollen polyester elastomer, and
   Wi is the weight of the initial (dry polymer).

The swelling ratio of the polyester elastomer (A) is suitably determined as follows.

A sample of polyester elastomer (A) without solvent or emollient, or a sample of polyester elastomer with solvent or emollient can be used. In case a sample of polyester elastomer (A) with solvent or emollient is used, the weight of the solvent or emollient already present in the composition is considered and subtracted from the initial sample weight Wi.

The swelling procedure is carried out at ambient temperature (23° C.).

About 1.9-2.1 grams of the polyester elastomer is placed in a 25 mL beaker. In the same beaker the polyester elastomer is mixed with 24.9-25.1 grams of coco-caprylate/caprate as solvent. The polyester elastomer is allowed to disperse and absorb (swell) the solvent for 30 minutes. The weight of the filter component (such as Thermo Scientific™ Nalgene™ Rapid-Flow™ Sterile Disposable Filter Units) is determined. After the polyester elastomer has swelled, the mixture in the beaker is mixed and poured into the filter. The beaker is rinsed with about 4.9-5.1 grams of coco-caprylate/caprate solvent to complete transfer of the swelled polyester elastomer. The excess solvent in the gel mixture is allowed to pass through the filter. The filter with swollen polyester elastomer is weighed when no excess solvent is observed on its surface (which can take about 4-18 hours) to give Ws.

The swelling ratio (SR) is calculated by the equation above.

In an aspect, the swelling ratio of the elastomer (A) is from about 1 gram/gram to about 15 gram/gram, from about 1 gram/gram to about 5 gram/gram, from about 1 gram/gram to about 4 gram/gram, from about 1 gram/gram to about 2 gram/gram. In some embodiments, the swelling value of the elastomer is about 15 gram/gram, about 14 gram/gram, about 13 gram/gram, about 12 gram/gram, about 11 gram/gram, about 10 gram/gram, about 9 gram/gram, about 8 gram/gram, about 7 gram/gram, about 6 gram/gram, 5 gram/gram, about 4.8 gram/gram, about 4.6 gram/gram, about 4.4 gram/gram, about 4.2 gram/gram, about 4 gram/gram, about 3.8 gram/gram, about 3.6 gram/gram, about 3.4 gram/gram, about 3.2 gram/gram, about 3 gram/gram, about 2 gram/gram, or about 1 gram/gram.

### Polyester Elastomer Component (A) Used as a Gel

As mentioned before in an aspect, a polyester elastomer composition (A) is prepared by shearing the polyester elastomer with a solvent or emollient, as described herein, to form a sheared polyester elastomer gel. In another aspect, a polyester elastomer gel is prepared by combining the polyester elastomer, as described herein, with a solvent or emollient, as described herein, thereby forming a mixture and shearing the mixture.

In an aspect, the shear force is provided by any type of mixing and shearing equipment. In an aspect, the mixing and shearing equipment is batch mixer, planetary mixer, single or multiple screw extruder, dynamic or static mixer, colloid mill, homogenizer, sonolator, three roll mill, or a combination thereof.

Subjecting these polyester elastomer compositions to a shearing force produces a polyester elastomer gel (A) which is particular suitable for use in the personal care or cosmetic applications that has an improved spreadability and an improved substance or feel. The claimed cosmetic or personal care applications where this property is most desirable included, but is not limited to, can have for example the form of skin creams, facial creams, hair care products such as shampoos, conditioners, mousses, and styling gels, protective creams, color cosmetics such as lipsticks, foundations, blushes, makeup, and mascara, and other cosmetic formulations.

In an aspect, the viscosity of the polyester elastomer (A) used in gel form is from about 10 cp to about 1,000,000 cp as measured by rheometer at a shear rate of 0.1 s−1. In an aspect, the viscosity of the gel at 25° C. is from about 30,000 cp to about 900,000 cp. In an aspect, the viscosity of the gel is about 10 cp, about 1,000 cp, about 5,000 cp, about 10,000 cp, about 15,000 cp, about 20,000 cp, about 25,000 cp, about 30,000 cp, about 35,000 cp, about 40,000 cp, about 45,000 cp, about 50,000 cp, about 55,000 cp, about 60,000 cp, about 65,000 cp, about 70,000 cp, about 75,000 cp, about 80,000 cp, about 85,000 cp, about 90,000 cp, about 95,000 cp, about 100,000 cp, about 150,000 cp, about 200,000 cp, about 250,000 cp, about 300,000 cp, about 350,000 cp, about 400,000 cp, about 450,000 cp, —40—about 500,000 cp, about 550,000 cp, about 600,000 cp, about 650,000 cp, about 700,000 cp, about 750,000 cp, about 800,000 cp, about 850,000 cp, about 900,000 cp, about 950,000 cp, or about 1,000,000 cp.

The viscosity of the polyester elastomer gel is measured by Anton Paar rheometer MCR 301, with probe PP25/S at gap 1 mm. The measuring profile is to use flow curve with shear rate 0.01-100/s under temperature 25° C. In measurement, a sample is loaded onto the rheometer stage, the probe is lowered, and the sample is allowed to equilibrate for 3 minutes, after which the test is performed. The viscosity at 10/s is reported.

In an aspect, the polyester elastomer (A) used as gel is comprised of particles of size from about 1 μm to about 500 μm as measured by a laser diffraction particle size analyzer. In an aspect, the gel is comprised of particles of size from about 20 μm to about 400 μm. In an aspect, the gel is comprised of particles of size of about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 125 μm, about 150 am, about 175 μm, about 200 μm, about 225 μm, about 250 μm, about 275 μm, about 300 μm, about 325 μm, about 350 μm, about 375 μm, or about 400 μm.

In an aspect, the polyester elastomer (A) used as gel is comprised of particles of size with D10 between 10 m to 50 μm, D50 between 20 m to 100 μm, and D90 between m to 200 μm.

The size of particles in polyester elastomer (A) used as gel is suitably measured by HORIBA Scientific Partica LA-960 Laser Scattering Particle Size Analyzer. The sample is prepared by blending 0.3 g of elastomer gel (A) with 15 g of coco-caprylate/caprate and mixing thoroughly at 23° C. A few drops of the diluted gel sample are transferred into the cuvette containing neat coco-caprylate/caprate while constantly stirring. Once the transmittance reaches the acceptable range, the measurement is performed at 23° C. Particle size values of D10, D50 and D90 are reported. The parameter D10 signifies the point in the size distribution, up to and including which, 10% of the total volume of material in the sample is ‘contained’. The parameter D50 signifies the point in the size distribution, up to and including which, 50% of the total volume of material in the sample is ‘contained’. The parameter D90 signifies the point in the size distribution, up to and including which, 90% of the total volume of material in the sample is ‘contained’.

Polyester elastomer (A) as a gel according to the present invention are characterized by the oscillation amplitude as well as oscillation frequency-dependent rheology tests at 25° C. In the linear viscoelastic region within the frequency range from 0.01-100 Hz, a gel has a storage modulus G′ which is always greater than the loss modulus G″. G′ and G″ here are rheological parameters known to the person skilled in the art. The elastic or storage modulus, designated as G′, is an indicator of how elastic the material is i.e., how much mechanical energy is being stored per cycle of deformation whereas, the viscous or loss modulus, namely G″, is the measure of the lost or dissipated mechanical energy as heat and/or other form per cycle of deformation and they collectively quantify the elastic or viscous fraction of viscoelastic solids and/or liquids and are described for example in Ferry, J. D., Viscoelastic Properties of Polymers, John Wiley & Sons, Inc. New York, 1980.

The polyester elastomer (A) as gels used according to the invention have an excellent yield point which, for example, has an advantageous effect on their thickening properties and also their ability to stabilize dispersed constituents of personal care formulations. For dynamic oscillation rheology tests, for example a MCR 301 Rheometer (Anton Paar, Graz, Austria) equipped with a 25 mm parallel plate steel geometry can be used.

Polyester elastomer (A) used as gels are notable for the fact that, at a shear rate of 1 l/s and a temperature of 25° C., they have a viscosity of less than 100,000,000 cp and at the same time satisfies G′>G″; Tan-8<1 within the linear viscoelastic region demonstrating a frequency nearly invariant characteristics. The polyester gels prepared by the methods described herein are characterized by good flowability, which has an advantageous effect on their handleability and processability, but nevertheless have a pronounced yield point and therefore good thickening and stabilizing properties.

In an aspect, the storage modulus (G′) of the polyester elastomer (A) used as gel is from about 10 Pa to about 100,000 Pa as measured by rheometer within linear viscoelastic region using dynamic—42—rheology. In an aspect, the storage modulus (G′) of the gel is from about 100 Pa to about 50,000 Pa. In an aspect, the storage modulus (G′) of the gel is from about 500 Pa to about 30,000 Pa, In an aspect, the storage modulus (G′) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

In an aspect, the loss modulus (G″) of the polyester elastomer (A) used as gel is from about 10 Pa to about 100,000 Pa as measured by rheometer within linear viscoelastic region using dynamic rheology. In an aspect, the loss modulus (G″) of the gel is from about 100 Pa to about 50,000 Pa. In an aspect, the loss modulus (G″) of the gel is from about 500 Pa to about 30,000 Pa, In an aspect, the loss modulus (G″) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

The storage modulus G′ and loss modulus G″ of the polyester elastomer (A) used as gel are measured by Anton Paar rheometer MCR 301, with probe PP25/S at gap 1 mm. The measuring profile is to use flow curve with shear rate 0.01-100/s under temperature 25° C. The measuring profile is to use amplitude sweep with oscillatory strain 0.001-100%, with frequency 1 Hz and under temperature 25° C. In measurement sample is loaded onto the rheometer stage, the probe is lowered and the sample is allowed to equilibrate for 3 minutes after which the amplitude sweep test is performed. The LVR region is determined and the respective G′ value is reported. In an aspect, the polyester elastomer composition is prepared using the methods described herein.

In an aspect, the polyester elastomers described herein are produced using the principles of green chemistry. In an aspect, the polyester elastomers described herein are produced by a simple, efficient environmentally friendly process, with no toxic raw materials used, and no toxic side products generated.

In an aspect, the polyester elastomer gels described herein are produced using the principles of green chemistry.

In an aspect, the polyester elastomer gels described herein are produced by a simple, efficient environmentally friendly process, with no toxic raw materials used, and no toxic side products generated.

### Hair Care Compositions

The hair care compositions of the invention are used to treat the hair and/or the scalp in particular of humans, in particular, for cosmetic purposes, i.e. they are preferably cosmetic hair care compositions.

Hair care compositions in accordance with the invention are suitably designed to serve a variety of functions, e.g. from cleansing and conditioning to protecting and styling the hair and from cleansing, protecting and treating the scalp. Different compositions may be formulated to address specific hair concerns or goals. Below is a non-exhaustive albeit detailed list of the functions of hair care formulations of the invention, broken down by categories and functions.

### 1. Cleansing

Shampoos: Shampoos are primarily designed to cleanse the scalp and hair by removing dirt, oil, and styling product buildup. They often contain surfactants (detergents) like sodium lauryl sulfate (SLS) or milder alternatives like sodium coco-sulfate.
Clarifying Shampoos: These are stronger formulations designed to remove heavy buildup from styling products, hard water minerals, or chlorine, often containing stronger cleansing agents.
Dry shampoos: Dry shampoos absorb the greasiness from hair without the need of water. They are often used to prolong the period in-between washes, extending the “fresh” appearance.

### 2. Conditioning

Conditioners: These products are designed to add moisture, smooth the hair cuticle, and improve the overall texture and manageability of the hair. They often contain emollients, silicones, proteins, or humectants like glycerin and panthenol.
Leave-in Conditioners: These products provide ongoing moisture and protection throughout the day, often formulated with lighter, non-greasy emollients.
Deep Conditioners (Hair Masks): Deep conditioners provide intense hydration and repair, using heavier oils (e.g., argan oil, coconut oil) or proteins (e.g., keratin, silk proteins) to nourish and strengthen the hair.
Protein Treatments: These formulations are specifically designed to repair and strengthen hair by replenishing lost protein, which is essential for hair structure and resilience.

### 3. Moisturizing

Moisturizing Shampoos and Conditioners: These are designed to provide hydration to dry or frizzy hair, typically containing humectants, oils, or glycerin.
Oils & Serums: Lightweight or heavier oils like argan, jojoba, or macadamia are often used to seal in moisture and add shine. These products smooth the cuticle, reducing frizz and enhancing texture.

### 4. Detangling

Detanglers and Leave-in Conditioners: These products contain ingredients that help to reduce static, improve slip, and make it easier to comb through wet or dry hair without causing breakage.
Silicones: Many detanglers contain silicones like dimethicone or cyclopentasiloxane, which provide a smooth, slippery coating on hair strands to prevent tangling and minimize friction.

### 5. Shine Enhancement

Shine Serums and Glosses: These products typically contain light-reflecting agents, oils, or silicones that smooth the hair surface and create a glossy, healthy appearance.
Polymers: Certain polymers can be added to hair products to help enhance the light-reflecting properties of hair, giving it a shiny, polished look.

### 6. Volume and Thickness

Volumizing Shampoos and Conditioners: These formulations often contain ingredients that swell hair fibers or provide lightweight conditioning to avoid weighing down the hair. Ingredients like hydrolyzed wheat proteins or cellulose can be used to thicken the hair shaft.
Root Lifting Sprays and Mousses: These styling products provide volume by lifting hair from the roots, often using lightweight polymers or starch derivatives.
Thickening Sprays and Gels: Designed to coat the hair fibers and create the appearance of thicker hair, often formulated with ingredients like silica or protein derivatives.

### 7. Frizz Control and Smoothing

Anti-Frizz Shampoos and Conditioners: These products are formulated to smooth the cuticle and prevent moisture from entering or escaping the hair, thus minimizing frizz. Common ingredients include silicones (e.g., dimethicone, cyclopentasiloxane), oils, and butters (e.g., shea butter).
Anti-Frizz Serums and Creams: These are applied to damp or dry hair and coat the strands to reduce frizz and provide a smoother, sleeker finish.
Keratin Treatments: These products are specifically designed to smooth the hair by infusing it with keratin, a protein that reinforces hair structure.

### 8. Hair Repair and Strengthening

Protein Treatments: Formulations containing hydrolyzed proteins (e.g., keratin, silk, collagen, or wheat protein) are designed to rebuild and strengthen damaged hair, restoring elasticity and reducing breakage.
Bond-Repair Treatments: Products like Olaplex use bond-building technology to help restore the disulfide bonds in the hair, particularly useful after chemical treatments like coloring or bleaching.
Reconstructive Shampoos and Conditioners: These are targeted at severely damaged hair, using stronger proteins and reparative ingredients to improve hair strength and elasticity.

### 9. Scalp Care

Scalp Shampoos and Exfoliants: These products cleanse the scalp and remove dead skin cells, excess oil, and product buildup. Ingredients like salicylic acid, tea tree oil, or menthol are commonly used for their clarifying and refreshing properties.
Scalp Treatments: These are used to address scalp issues like dandruff, dryness, or irritation, often containing active ingredients like zinc pyrithione (for dandruff), caffeine (for stimulating hair growth), or essential oils (like peppermint or rosemary).
Anti-Dandruff Shampoos: These formulations contain active agents such as ketoconazole, selenium sulfide, or zinc pyrithione to treat fungal-related dandruff and flakiness.

### 10. Color Care

Color-Protecting Shampoos and Conditioners: These formulations are designed to protect hair color from fading, often containing UV filters, antioxidants, and gentle surfactants to avoid stripping the color.
Color-Enhancing Products: These products help to enhance or refresh color-treated hair, including purple shampoos for blondes (to tone yellow or brassy tones) and color-depositing conditioners for brunettes and redheads.
Hair Dyes and Colorants: These products provide permanent, semi-permanent, or temporary color to the hair, using either oxidative or direct dyeing methods.

### 11. Sun Protection

UV Protectant Sprays and Leave-ins: These products are formulated with UV filters and antioxidants to protect hair from the damaging effects of the sun, which can cause dryness, fading, and brittleness.
Sunscreen Infused Shampoos and Conditioners: Some hair care formulations include sunscreen agents (e.g., avobenzone, benzophenone) to provide built-in UV protection for both the scalp and hair.

### 12. Hair Styling

Hair Gels, Mousses, and Foams: These products help shape and define hairstyles, providing varying levels of hold and volume. Ingredients like polyvinylpyrrolidone (PVP) or acrylates are often used for their ability to form a flexible, strong hold.
Hair Sprays: These provide hold for styles, helping them stay in place. They may range from light to strong hold, depending on the desired effect. Typically contain polymers like acrylates or copolymers to provide flexibility.
Pomades and Waxes: These products add texture, definition, and shine to the hair, and are often used for creating sleek or messy looks. They contain heavier oils or waxes like beeswax, carnauba wax, or lanolin.
Creams and Pastes: These provide a more natural finish and are often used for controlling texture and adding volume without stiffness.

### 13. Curl Defining

Curl Creams and Gels: These formulations enhance natural curls or waves, offering definition, moisture, and frizz control. Ingredients may include silicones, humectants like glycerin, or natural oils.
Curl Activators: These products are designed to enhance curl formation, offering hold without crunchiness. They are usually lightweight and may contain ingredients like proteins or hydrating agents.
Anti-Frizz Curl Creams: These products tame frizz while maintaining curl shape, often using ingredients that smooth the hair and provide moisture retention.

### 14. Heat Protection

Heat Protectant Sprays and Serums: These formulations are designed to create a barrier between the hair and styling tools (flat irons, curling irons, blow dryers). They typically contain silicones or polymers that help prevent heat damage.
Thermal Protectants: These are more intensive treatments that protect hair from high heat styling, often used before using hot tools on vulnerable hair (especially after chemical treatments).

Additional hair care active compounds (B) which are different from (A) (and (C) and (D)) are ingredients specifically designed to target and improve specific hair and scalp concerns. These ingredients typically have functional roles such as moisturizing, strengthening, protecting, or treating hair-related conditions. Below is a non-exhaustive complete list of hair care active compounds (B) which can be used in the hair care compositions in addition to the crosslinked polyester component (A):

### 1. Moisturizing Agents/Humectants

These ingredients help attract moisture to the hair and scalp, keeping them hydrated.
Glycerin: A common humectant that draws moisture from the air to the hair.
Propylene Glycol: Draws moisture into the hair and scalp.
Butylene Glycol: Moisturizing and conditioning properties.
Sorbitol: A sugar alcohol used to attract moisture.
Panthenol (Pro-Vitamin B5): Adds moisture, improves hair elasticity, and enhances shine.
Hyaluronic Acid: A powerful humectant that retains moisture and prevents water loss.
Aloe Vera Extract: Known for its moisturizing and soothing properties.
Honey: A natural humectant that pulls moisture into the hair and retains it.
Urea: Hydrates and softens the hair and scalp. 2. Conditioning Agents

These help improve the texture, manageability, and softness of hair.
Behentrimonium Chloride: A gentle, cationic surfactant that smooths and detangles hair.
Cetrimonium Chloride: Improves texture and provides conditioning.
Silicones (Dimethicone, Cyclopentasiloxane, Amodimethicone): Help smooth the hair, reduce frizz, and enhance shine.
Polyquaternium-7, Polyquaternium-10: Silky-feeling cationic conditioners that help retain moisture and smooth hair.
Stearalkonium Chloride: A cationic conditioning agent that makes hair softer and more manageable.

### 3. Proteins

Proteins are vital for strengthening and repairing damaged hair, as they help to rebuild the hair structure.
Hydrolyzed Keratin: Restores strength and reduces breakage by filling in gaps in the hair shaft.
Hydrolyzed Silk Protein: Strengthens and enhances shine.
Hydrolyzed Collagen: Improves hair elasticity and moisture retention.
Wheat Protein (Hydrolyzed Wheat Protein): Strengthens hair, prevents damage, and adds body.
Soy Protein (Hydrolyzed Soy Protein): Helps with moisture retention and strengthens the hair.
Rice Protein (Hydrolyzed Rice Protein): Increases hair's volume and strengthens hair strands.

### 4. Lipids/Fatty Acids

Lipids help strengthen the hair cuticle and improve its smoothness.
Ceramides (Ceramide 1, Ceramide 2, Ceramide 3): Lipid molecules that help restore the hair's natural barrier, reduce frizz, and improve moisture retention.
Fatty Alcohols (Cetyl Alcohol, Stearyl Alcohol): These emulsifiers and emollients help improve texture and feel, providing softness to the hair.
Shea Butter: Rich in fatty acids, it deeply nourishes and protects hair from dryness.
Coconut Oil: A deeply moisturizing oil that penetrates the hair shaft and prevents protein loss.

### 5. Scalp Treatment Ingredients

These compounds are aimed at improving scalp health, reducing flakiness, and promoting hair growth.
Salicylic Acid: Exfoliates the scalp, removing dead skin cells and preventing dandruff.
Zinc Pyrithione: Known for its antifungal and antibacterial properties, commonly used to treat dandruff and seborrheic dermatitis.
Tea Tree Oil: An essential oil with antimicrobial properties that can help treat dandruff and soothe the scalp.
Ketoconazole: An antifungal agent used to treat dandruff and other scalp conditions.
Niacinamide (Vitamin B3): Improves the scalp's barrier function, reduces inflammation, and promotes healthy hair growth.
Menthol: Provides a cooling sensation and helps improve circulation to the scalp.
Biotin (Vitamin B7): Promotes hair growth and strengthens hair follicles.
Panthenol: Not only hydrates but also soothes the scalp and promotes healthy hair growth.
Aloe Vera: Soothes an irritated or dry scalp and promotes healing.
Ginseng Extract: Stimulates hair follicles and enhances scalp circulation to promote hair growth.

### 6. Hair Growth and Repair Agents

These compounds are designed to help with hair thinning, hair loss, and repair of damaged hair.
Minoxidil: Widely used in treatments for hair thinning and androgenic alopecia (male/female pattern baldness).
Biotin: Supports the growth of healthy hair by strengthening the hair follicles.
Caffeine: Stimulates hair growth by improving circulation to the scalp.
Finasteride: A prescription medication used to treat male pattern baldness by inhibiting DHT (dihydrotestosterone) production.
Saw Palmetto: An herbal extract believed to block DHT, a hormone linked to hair loss.
Copper Peptides: Stimulate hair growth and collagen production while helping to repair damaged hair.
Argan Oil: Rich in essential fatty acids and antioxidants, it promotes hair strength, elasticity, and growth.
Procapil: A combination of biotinyl-GHK, apigenin, and oleanolic acid, it is used to strengthen hair follicles and promote hair growth.
Apple Stem Cells: Promote the regeneration of hair follicles and protect against oxidative stress that could lead to hair loss.

### 7. Antioxidants

Antioxidants help protect hair from free radical damage (e.g., UV, pollution) and maintain overall hair health.
Vitamin E (Tocopherol): Prevents oxidative damage to the scalp and hair, adds shine, and strengthens hair.
Vitamin C: Reduces oxidative stress and promotes collagen production, which supports hair growth.
Green Tea Extract: Contains EGCG (epigallocatechin gallate), which helps protect hair from free radical damage and promotes healthy hair growth.
Ferulic Acid: A potent antioxidant that helps protect the hair and scalp from UV damage and environmental pollutants.
Resveratrol: Another antioxidant that helps prevent damage to hair from UV and pollution.

### 8. UV Protectants

These ingredients help protect hair from the damaging effects of UV rays.
Benzophenone-4: A UV filter that helps protect hair from sun damage.
Ethylhexyl Methoxycinnamate (Octinoxate): Absorbs UV rays, preventing sun damage to the hair.
Homosalate: Used in hair care products to protect hair from UV damage.
Tinosorb S: A broad-spectrum UV filter that provides protection against both UVA and UVB rays.

### 9. Dandruff Treatment Compounds

These ingredients help treat the symptoms of dandruff and flakes.
Pyrithione Zinc: An active ingredient with antifungal properties, commonly used to treat dandruff.
Tar Extract: Used in shampoos for treating dandruff, seborrheic dermatitis, and psoriasis.
Selenium Sulfide: A compound that reduces scalp flakiness and prevents dandruff.
Climbazole: A mild antifungal ingredient that helps treat dandruff.

### 10. Volumizing Agents

These compounds are used to add volume to the hair, particularly for fine or limp hair.
Polyquaternium-11: Adds volume, thickness, and shine.
Panthenol: Adds thickness and body to the hair while also providing moisture.
Hydrolyzed Wheat Protein: Strengthens hair and adds volume by increasing the hair shaft's thickness.

### 11. Anti-Frizz Agents

These ingredients smooth the hair shaft and reduce the appearance of frizz.
Dimethicone: A type of silicone that smooths the hair and provides anti-frizz benefits.
Amodimethicone: A silicone that conditions the hair and controls frizz.
Polyquaternium-10: A conditioning agent that adds smoothness and reduces frizz.
Triticum Vulgare (Wheat) Germ Oil: Reduces frizz and improves the moisture balance of the hair.

### 12. Chelating Agents

These compounds are used to bind and remove mineral buildup (e.g., from hard water or styling products) from the hair.
EDTA (Ethylene Diamine Tetraacetic Acid): A chelating agent that binds to minerals and prevents their buildup on hair.
Citric Acid: Often used to adjust pH levels and help remove mineral buildup.

The present disclosure relates to hair care, i.e. personal hair care formulations comprising at least one crosslinked polyester component (A), optionally at least one cosmetically acceptable carrier (C) each as described above, and optionally one or more other components commonly used in the hair care field (B), different from components (A) or (C) and further optional one or more other components commonly used in the cosmetic field (D) different from (A) to (C). In an aspect the hair care formulations may also provide therapeutical benefits to patients in particular depending on the kind of component (B), thereby preventing or treating e.g. hair or scalp diseases. But preferably the hair care formulations are for cosmetic purposes for the treatment of hair or scalp of humans or animals, preferably humans.

In some aspects, the other components commonly used in the cosmetic field (D) different from components (A), (B) and (C) are selected from the group pigments, water, emollients, fatty acids, fatty alcohols, film formers, emulsifiers, thickeners, preservatives, rheology modifiers, pH adjustors, reducing agents, foaming agents, de-foaming agents, chelating agents, gums, thickeners, oils, waxes, fragrances, essential oils, and combinations thereof.

In an aspect the hair care compositions do not contain any colorant including pigment or the like.

In an aspect the hair care compositions do not contain any UV filter compound.

In an aspect the hair care compositions do neither contain any colorant nor any UV filter compound.

In an aspect the hair care compositions can comprise:
0.1 to 85%, preferably 4 to 80%, more preferably 6 to 50% by weight of one or more crosslinked polyester components (A),
0 to 80% by weight, preferably from 1 to 70% by weight, and more preferably from 2 to 60% by weight of one or more hair care active compounds (B),
0 to 90% by weight of one or more cosmetically acceptable carriers (C),
0 to 60% by weight of one or more other components commonly used in the cosmetic field (D),
based on the total weight of the composition.
   Specific Benefits of Using the Crosslinked Polyester Component (A), in Particular, when Used as a Gel in One or More Solvents or Emollients as Described Herein, in Hair Care Compositions

In the following the specific benefits of the hair care compositions of the invention are explained in more detail.

### BACKGROUND

Human hair is constantly exposed to damaging processes both physical and chemical. Physical processes that damage the hair include heat drying with a hair dryer or hot iron, brushing, and combing. Chemical processes include shampooing, permanent weaving, dyeing and bleaching. All of these processes result in morphological damage to the hair fibers reducing the natural suppleness and manageability.

Shampooing hair cleans by removing excess soil and sebum. However, the shampooing process has disadvantages. Shampooing can cause hair to be in a tangled and unmanageable state, especially when the hair is damaged. Shampooing can also cause hair dryness due to the removal of natural oils or other hair moisturizing materials. After shampooing, the hair can also suffer from a perceived loss of “softness”. Softness, of course, is a generally desirable attribute for many users of shampoo products. As is well known, many consumers apply a rinse-off conditioner to hair after shampooing. In this respect, hair shampoos generally contain surfactants that often leave the hair undesirably harsh, dull, and dry. In addition, shampooed hair often becomes tangled when wet and/or dry such that combing and brushing of the hair is hampered. Furthermore, especially in low humidity environments (e.g., an RH below 35% such as can be found in cold winter climates), the hair frequently exhibits poor electrostatic properties such that it is susceptible to “fly-away.” Thus, to address the conditioning properties of the hair, both in the wet and dry states, conditioner is applied to render the hair more manageable, to minimize fly away, static charge, and tangling of the hair, as well as to soften hair and facilitate combing.

The act of hair conditioning is often referred to as a generic process of applying one or more products to the hair, separately or in a mixture. The desired effect of applying a hair conditioner product is to alter the frictional and/or mechanical properties of the hair, thereby improving the feel, texture, appearance and manageability of hair and reducing the friction between strands of the hair to allow smoother brushing or combing. Hair conditioning materials can be applied to the hair when the hair is wet, for example during showering, or when the hair is dry. Most commonly, the term refers to the former. Hair conditioning materials are usually applied from a formulation comprising the conditioning materials in conjunction with a number of lipophilic ingredients.

### Friction

Friction is a crucial quality to measure in haircare, as it affects appearance and manageability of hair. When friction is high, hair can become more tangled, damaged, and frizzy. In contrast, when hair has low friction, it can look and feel smooth and silky. Evaluation of the friction of hair provides information about the condition of the hair, which can be affected by such things as the degree of cuticle lift, cuticle breakage, surface erosion, and deposition of materials on the hair surface.

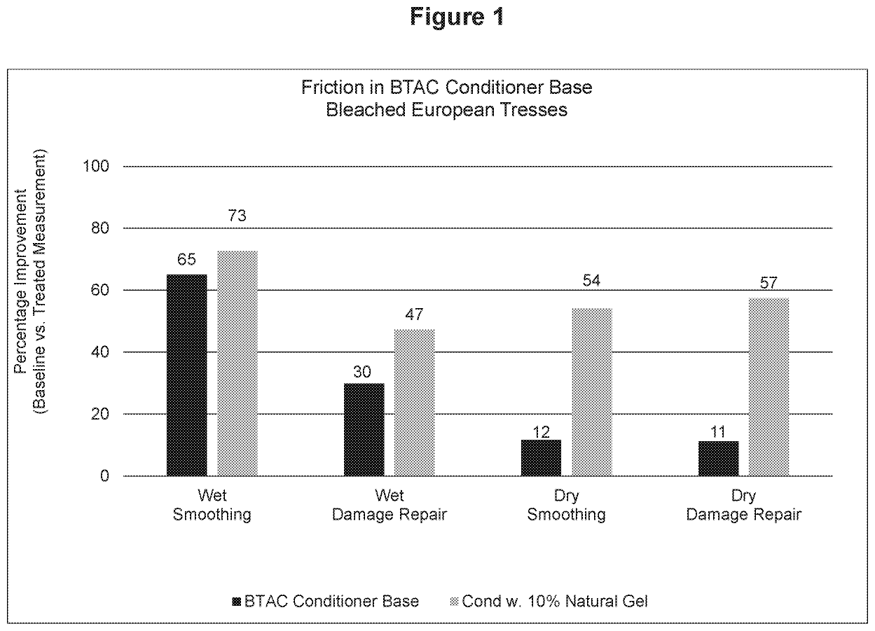

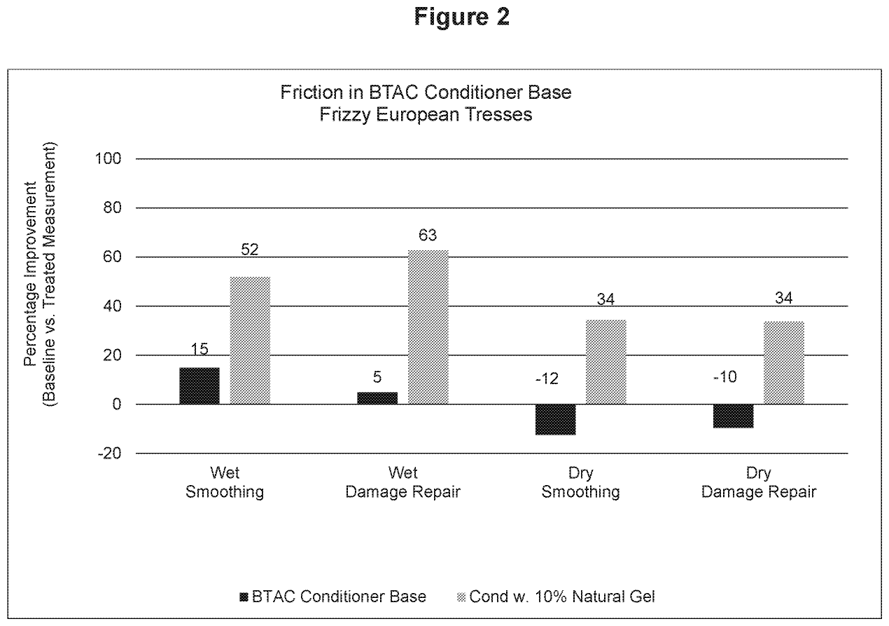

Lubricating agents are already known to prevent or minimize formation of split ends by reducing friction. The lubrication reduces the friction in the hair during combing and hence reduces the strength of the abrasive forces to which the hair is being subjected. This is turn reduces the number of entanglements during the combing process. When formulated into a BTAC-based hair conditioning composition (Example 9), crosslinked polyester component (A) in particular, when used as a gel in one or more solvents or emollients as described herein, reduces hair friction on both bleached European tresses (FIG. 1) and frizzy European tresses (FIG. 2). The crosslinked polyester component (A) in particular, when used as a gel in one or more solvents or emollients as described herein, improves smoothing of both dry and wet hair, and increases damage repair of both dry (after application) and wet hair, as seen in FIGS. 1 and 2. The conditioner base without any crosslinked polyester component (A) was tested in parallel as a control. Reduction in friction was evaluated by measuring the coefficient of friction using the Dia-Stron MTT175 Miniature Tensile Tester on frizzy European hair tresses.

### Anti-Frizz

Hair frizz is described by consumers as the appearance of unruly fibers at the top of the scalp and tips of hair as well as an increased volume through the bulk of the hair. Generally, they see this frizz on days where there is humid weather and the level of moisture in the air is high. The appearance of frizz is undesired, and it is often associated with a loss of shine and smoothness. Hair frizz is a problem that commonly occurs in all hair types, especially to people who have wavy and curly hair, particularly when humidity levels and/or dew points are high; this typically occurs in the summer months and at tropical latitudes.

What is normally thought of as “hair” (i.e., the hair shaft, external to the hair root under the skin) is comprised of keratin, a protein in which disulfide bonds hold the strands together. The hair shaft comprises three layers: the medulla, the cortex and the cuticle. The medulla is a thin core of transparent cells and air spaces. The cortex forms the bulk of the hair shaft and usually contains pigment and long keratin filaments, which are held together by disulfide and hydrogen bonds.

The cuticle is a protective layer comprised of overlapping cells, organized like fish scales facing downwards. The outer cuticle holds the hair shaft in the hair follicle by means of a bond. When it is healthy, it also minimizes the movement of water (moisture) in and out of the underlying cortex. However, chemical processes and weathering can lift the cuticle from the cortex and disrupt this balance.

When high humidity causes frizz, the cuticle layer of hair is raised. The hair cuticles normally regulate the water absorption in the hair. Raised cuticles occur in the areas of naturally curly hair in which there is a bend in the curl and permit moisture to be absorbed by the hair cortex. As a result, the hair may appear dry (because the cuticle may indeed be dry); the hair shaft increases in volume due to the water uptake by the cortex causing the hair to swell. In general, extremely wavy and curly hair tends to have more frizz than straight hair because the hair does not properly regulate the amount of water absorbed by the hair.

In addition to curly or wavy hair, hair that has been damaged by chemical processing (such as straightening, permanent waving, hair dyeing or bleaching), heat (such as by blow drying and the use of flat irons or curling irons), mechanical damage (such as by excessive combing and brushing) and environmental damage (such as sunlight, air or water pollution, chlorine, etc.) causes cuticle damage, permitting water to enter the cortex and the hair to absorb more moisture.

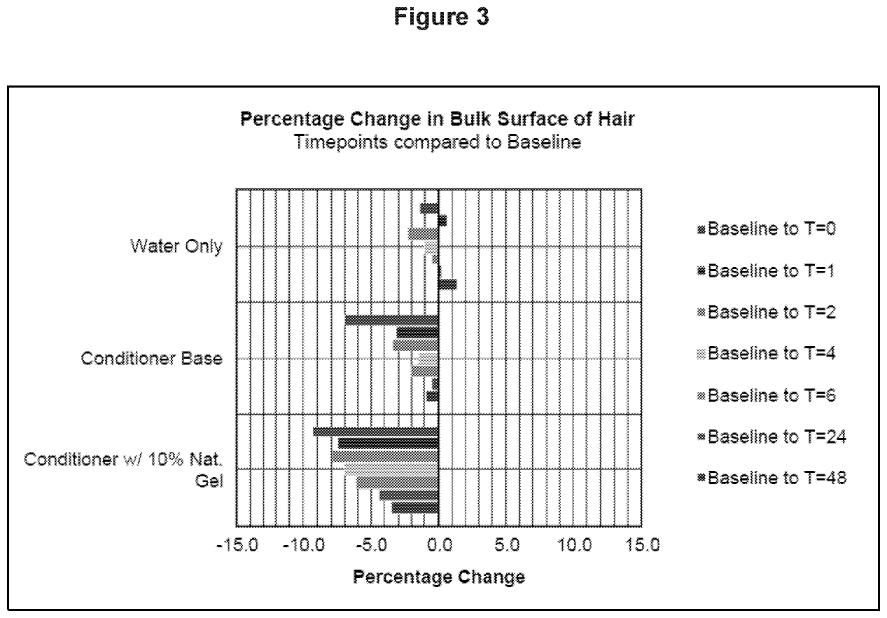

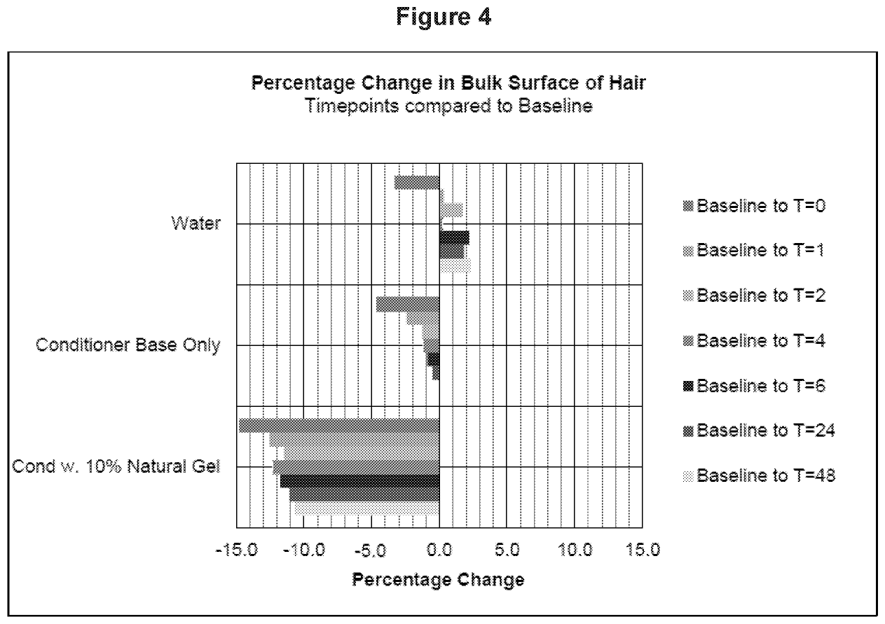

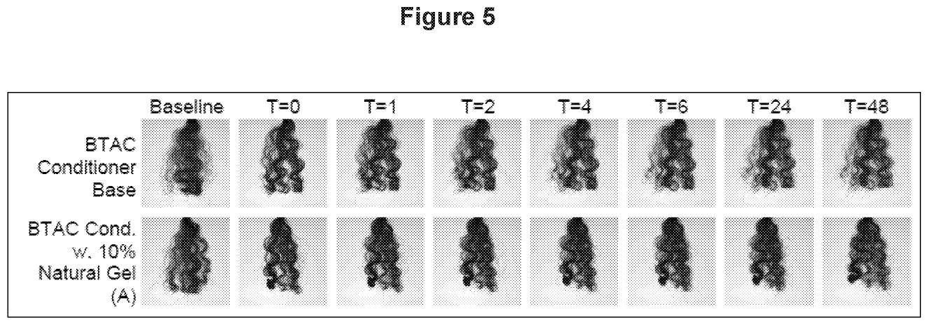

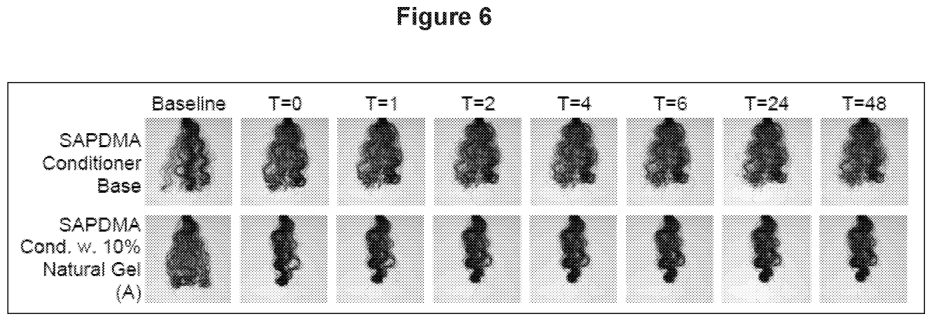

More recently, various conditioners and “leave-in” products have been formulated to control frizz. When formulated into a hair conditioning composition, crosslinked polyester component (A) in particular, when used as a gel in one or more solvents or emollients as described herein, reduces hair volume, which decreases frizz. This benefit was observed immediately, and as a long-lasting effect up to 48 hours, as measured in FIGS. 3 and 4, and as observed in photographs in FIGS. 5 and 6. The crosslinked polyester component (A) shows significant reduction in frizz on frizzy European hair tresses when used in both BTAC and SAPDMA based chassis. Water only and the conditioner base without any crosslinked polyester component (A) were tested in parallel as a control. Anti-frizz was measured through image analysis by the Bossa Nova Vision Bolero, and the study was conducted on frizzy European hair tresses.

### Sensory

Rinse-off hair care compositions typically comprise one or more conditioning agents to provide sensory benefits to the hair. Consumers desire hair products that feel light and are easy to apply. “Slip” from a hair care product increases application ease, increases combing ease, and prevents damage/breakage of hair. Slip refers to an increased lubrication of the hair fibers which reduces friction and tangling. Tangling can increase damage and breakage to the hair, and lead to higher combing force. Thus, prevention of tangling is essential to maintain healthy, smooth, and soft hair.

When formulated into a hair conditioning composition, crosslinked polyester component (A) in particular, when used as a gel in one or more solvents or emollients as described herein, increases slip in both the wet hair lengths and wet hair tips. In addition, crosslinked polyester component (A), when used as a gel in one or more solvents or emollients as described herein, increases ease of combing in both wet and dry hair when formulated into a hair conditioning composition. Smoothness of the hair lengths and tips were also noticeably improved with the addition of the crosslinked polyester component (A) to hair conditioners. The polyester component (A), when used as a gel in one or more solvents or emollients as described herein, led to an increase in de-tangling of the hair, further preventing hair damage when formulated into a hair conditioner.

It was observed that the crosslinked polyester component (A) led to an increase in curl definition and a reduction in volume of dry hair, when formulated into a hair conditioning composition. The conditioner base without any crosslinked polyester component (A) was tested in parallel as a control. Consumers with wavy or curly hair desire for their products to provide better curl definition and reduce frizz that often reduces the appearance of well-defined curls. Smoothing, detangling, and lubricating properties were evaluated by an expert sensory panel, by measuring the combing force, and by measuring the coefficient of friction, through the Dia-Stron MTT175 Miniature Tensile Tester. These studies were run on bleached or frizzy European hair tresses.

The invention thus also relates to the use of at least one crosslinked polyester component (A), comprising a crosslinked polyester which is reaction product of:
(i) at least one compound selected from a polycarboxylic acid, a poly-carboxylic acid ester, and combinations thereof,
(ii) at least one polyol, and
(iii) optionally one or more monofunctional component selected from
   (a) a mono-carboxylic acid; and
   (b) a mono-alcohol,
for the preparation of hair care compositions.

In an embodiment the at least one crosslinked polyester component (A), is provided with the hair care composition as a flowable gel composition comprising at least one solvent or emollient.

In an embodiment the invention also relates to the use of at least one crosslinked polyester component (A), comprising a crosslinked polyester which is reaction product of:
(i) at least one compound selected from a polycarboxylic acid, a poly-carboxylic acid ester, and combinations thereof,
(ii) at least one polyol, and
(iii) optionally one or more monofunctional component selected from
   (a) a mono-carboxylic acid; and
   (b) a mono-alcohol, for the preparation of hair care composition.

In an embodiment the invention also relates to the use of at least one crosslinked polyester component (A), comprising a crosslinked polyester which is reaction product of:
(i) at least one compound selected from a polycarboxylic acid, a poly-carboxylic acid ester, and combinations thereof,
(ii) at least one polyol, and
(iii) optionally one or more monofunctional component selected from
   (a) a mono-carboxylic acid; and
   (b) a mono-alcohol,
as hair conditioning agent in hair care compositions, and/or
as hair frizz controlling agent in hair care compositions, and/or
as hair smoothing agent in hair care compositions, such as dry hair smoothing agent, and/or
as hair detangling agent in hair care compositions, and/or
as hair friction reducing agent in hair care compositions, and/or
as hair combing facilitating agent in hair care compositions, and/or
as hair repairing agent in hair care compositions, and/or
as hair split ends reducing agent in hair care compositions, and/or
as hair volume controlling agent such as hair volume- or bulk surface-reducing agent in hair care compositions, and/or
as hair thickness-increasing agent in hair care compositions, and/or
as hair strengthening agent in hair care compositions, and/or
as hair colour protection agent in hair care compositions, and/or
as hair shine enhancing agent in hair care compositions, and/or
as hair moisturizing and hydrating conditioner in hair care compositions, and/or
as hair deep conditioner in hair care compositions, such as hair masks, and/or
as hair hold conditioner in hair care compositions, and/or
as hair curl defining agent in hair care compositions, and/or
as hair cleansing conditioner in hair care compositions.

In an embodiment the hair care compositions are selected from leave-in conditioners, rinse-out or rinse-through conditioners, hair styling compositions, such as hair sprays, gels, creams, mousses, lotions, pastes, pomades, waxes and foams, and hair masks.

In an embodiment the invention also relates to a method for
hair conditioning, and/or
hair frizz controlling, and/or
hair smoothing, such as dry smoothing, and/or
hair detangling, and/or
hair friction reducing, and/or
hair combing facilitating, and/or
hair repairing, and/or
hair split ends reducing, and/or
hair volume controlling, such as hair volume or bulk surface-reducing, and/or
hair thickness-increasing, and/or
hair strengthening, and/or
hair colour protecting, and/or
hair shine enhancing, and/or
hair moisturizing and hydrating, and/or
hair deep conditioning, and/or
hair hold conditioning, and/or
hair curl defining, and/or
hair cleansing conditioning,
comprising the step of adding at least one crosslinked polyester component (A), comprising a crosslinked polyester which is reaction product of:
(i) at least one compound selected from a polycarboxylic acid, a poly-carboxylic acid ester, and combinations thereof,
(ii) at least one polyol, and
(iii) optionally one or more monofunctional component selected from
   (a) a mono-carboxylic acid; and
   (b) a mono-alcohol,
to hair care compositions and applying said hair care compositions to hair.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Esterification to Prepare Polyester Elastomer Component (A)

In a suitable vessel equipped with agitation, heat, and an ability to distill off water, 130 g dimer acid was added along with 190 g hydrogenated castor oil. Next 50 g squalane was added as solvent. After all ingredients were charged under agitation, the temperature of the mass was raised to 180° C., and water was stripped off as formed. The temperature was held until gelation took place and polymer elastomer was formed. Thereafter the elastomer was broken into a powder by mechanical stirring.

### Example 2: Esterification to Prepare Polyester Elastomer Component (A)

In a suitable vessel equipped with agitation, heat, and an ability to distill off water, 200 g dimer acid was added along with 35 g diglycerol and 30 g oleic acid. Next 30 g squalane was added as solvent. After all ingredients were charged under agitation, the temperature of the mass was raised to 160° C., and water was stripped off as formed. The temperature was held until gelation took place and polymer elastomer was formed. Thereafter the elastomer was broken into a powder by mechanical stirring.

### Example 3: Esterification to Prepare Polyester Elastomer Component (A)

In a suitable vessel equipped with agitation, heat, and an ability to distill off water, 130 g hydrogenated dimer acid was added along with 190 g hydrogenated castor oil. Next 50 g squalane was added as solvent. After all ingredients were charged under agitation, the temperature of the mass was raised to 180° C., and water was stripped off as formed. The temperature was held until gelation took place and polymer elastomer was formed. Thereafter the elastomer was broken into a powder by mechanical stirring.

### Example 4: Esterification to Prepare Polyester Elastomer Component (A)

In a suitable vessel equipped with agitation, heat, and an ability to distill off water, 130 g hydrogenated dimer acid was added along with 70 g hydrogenated castor oil and 60 g dilinoleyl alcohol. Next 170 g squalane was added as solvent. After all ingredients were charged under agitation, the temperature of the mass was raised to 180° C., and water was stripped off as formed. The temperature was held until gelation took place and polymer elastomer was formed. Thereafter the elastomer was broken into a powder by mechanical stirring.

### Example 5: Preparation of Polyester Gel (A) from Polyester Elastomer

After an elastomer powder was produced, such as that made in Example 1, which did not separate upon standing, 32.5 g coco-caprylate/caprate was added to 175 g elastomer. After the elastomer and solvent mixture sit and swell for 1 hour, it was milled with a 3-roll mill, and then diluted with 300 g coco-caprylate/caprate. The milled elastomer solvent mixture was then homogenized in the reactor vessel to produce a creamy, translucent gel of very smooth consistency, suitable for use in personal care formulations.

### Example 6: Preparation of Polyester Gel (A) from Polyester Elastomer

After an elastomer powder was produced, such as that made in Example 4, which did not separate upon standing, 7.5 g coco-caprylate/caprate was added to 25 g elastomer. After the elastomer and solvent mixture sit and swell for 1 hour, it was milled with a 3-roll mill, and then diluted with 325 g coco-caprylate/caprate. The milled elastomer solvent mixture was then homogenized in the reactor vessel to produce a creamy, translucent gel of very smooth consistency, suitable for use in personal care formulations.

### Example 7: Preparation of Polyester Gel from Polyester Elastomer

After an elastomer powder was produced, such as that made in Example 1, which did not separate upon standing, 330 g coco-caprylate/caprate was added to 175 g elastomer. After the elastomer and solvent mixture sit and swell for 1 hour, it was mixed via homogenizer to produce a creamy, translucent gel of very smooth consistency, suitable for use in personal care formulations.

### Example 8: Preparation of Polyester Gel from Polyester Elastomer

After an elastomer powder was produced, such as that made in Example 4, which did not separate upon standing, 332.5 g coco-caprylate/caprate was added to 25 g elastomer. After the elastomer and solvent mixture sit and swell for 1 hour, it was mixed via homogenizer to produce a creamy, translucent gel of very smooth consistency, suitable for use in personal care formulations.

### Example 9: BTAC Hair Chassis Conditioning Formulation

| | | | With 10% |
|---|---|---|---|
| | | | polyester |
| | | Conditioner | elastomer gel (A) |
| | | Base | (Example 6) |
| Phase | INCI Name | % w/w | % w/w |
| A | Aqua (Water) | Up to 100 | Up to 100 |
| A | Disodium EDTA | 0.20 | 0.20 |
| B - Premix | Glycerin | 1.50 | 1.50 |
| B - Premix | Hydroxyethyl Cellulose | 1.50 | 1.50 |
| C | Citric Acid | 0.33 | 0.33 |
| D | Cetearyl Alcohol | 0.50 | 0.50 |
| D | Stearyl Alcohol | 0.50 | 0.50 |
| D | Behentrimonium | 0.20 | 0.20 |
| | Chloride (BTAC) | | |
| | (and) Isopropyl | | |
| | Alcohol | | |
| D | Elastomer Gel (A) | — | 10.00 |
| | of Example 6 | | |
| E | Phenoxyethanol | 1.00 | 1.00 |
| | (and) Benzyl | | |
| | Alcohol | | |
| | (and) Potassium | | |
| | Sorbate (and) Aqua | | |
| | (and) Tocopherol | | |
| E | Citric Acid | As required | As required |
| | pH/Viscosity cP | 3.88/13,100 | 3.89/18,700 |
| | (Brookfield | | |
| | Helipath T Bar C, | | |
| | 10 rpm, 1 min) | | |

### Preparation Protocol:

Add phase A to the vessel and stir until dissolved. Premix phase B and add to phase A. Begin heating slowly to 80-85° C.* with adequate stirring to prevent burning of the gel. Batch should thicken slightly at −50° C. Once heated to 80-85° C. add phase C. In a separate vessel prepare phase D by heating to 80-85° C. stir continuously throughout until all components are melted and homogeneous. Check both phases are at the same temperature (80-85° C. Add phase D to phase A then homogenise, until the formulation is white and smooth. Once the mixture is below 40° C., add phase E. Check viscosity and pH, adjust pH if necessary.

### Example 10: SAPDMA Hair Chassis Conditioning Formulation

| | | | With 10% |
|---|---|---|---|
| | | | polyester |
| | | Conditioner | elastomer gel (A) |
| | | Base | (Example 6) |
| Phase | INCI Name | % w/w | % w/w |
| A | Aqua (Water) | Up to 100 | Up to 100 |
| A | Disodium EDTA | 0.20 | 0.20 |
| B - Premix | Glycerin | 0.50 | 0.50 |
| B - Premix | Hydroxyethyl Cellulose | 0.50 | 0.50 |
| C | Citric Acid | 0.15 | 0.15 |
| D | Cetearyl Alcohol | 4.00 | 4.00 |
| D | Stearyl Alcohol | 4.00 | 4.00 |
| D | Stearamidopropyl | 1.00 | 1.00 |
| | Dimethylamine | | |
| | (SAPDMA) | | |
| D | Glyceryl Stearate | 1.00 | 1.00 |
| D | Elastomer Gel (A) | — | 10.00 |
| | of Example 6 | | |
| E | Phenoxyethanol | 1.00 | 1.00 |
| | (and) Benzyl Alcohol | | |
| | (and) Potassium | | |
| | Sorbate (and) Aqua | | |
| | (and) Tocopherol | | |
| E | Citric Acid | As required | As required |
| | pH/Viscosity cP | 3.88/13,100 | 3.89/18,700 |
| | (Brookfield | | |
| | Helipath T Bar C, | | |
| | 10 rpm, 1 min) | | |

### Preparation Protocol:

Add phase A to the vessel and stir until dissolved. Premix phase B and add to phase A. Begin heating slowly to 70-75° C.* with adequate stirring to prevent burning of the gel. Batch should thicken slightly at −50° C. Once heated to 70-75° C. add phase C. In a separate vessel prepare phase D by heating to 70-75° C. stir continuously throughout until all components are melted and homogeneous. Check both phases are at the same temperature (70-75° C. Add phase D to phase A then homogenise, until the formulation is white and smooth. Once the mixture is below 40° C., add phase E. Check viscosity and pH, adjust pH if necessary.

### Example 11: Leave-In Hair Serum

| INCI Name | % w/w |
|---|---|
| Aqua (Water) | Up to 100 |
| Hydroxypropyl Starch Phosphate | 1.80 |
| Disodium EDTA | 0.05 |
| Cetearyl Alcohol | 2.30 |
| Glyceryl Stearate (and) PEG-100 | 0.70 |
| Stearate | |
| C13-15 Alkane | 3.00 |
| Elastomer Gel (A) Example 6 | 5.00 |
| Behentrimonium Chloride | 0.50 |
| (and) Isopropyl Alcohol | |
| Aqua (and) Amodimethicone (and) | 1.00 |
| Trideceth-6 (and) Trideceth-12 | |
| (and) Glycerin | |
| Preservative | q.s. |
| Appearance: | Smooth, white emulsion |
| Viscosity: | ~15,000 cP (Brookfield Helipath, |
| | T Bar B, 10 rpm) |
| pH: | 5.0-6.0 |

### Preparation Protocol:

Add water to the main vessel and begin stirring. Add EDTA and allow to dissolve. Sprinkle Hydroxypropyl Starch Phosphate into a vortex and allow to disperse. Begin heating to 70° C. Add Phase B ingredients to a separate vessel and begin heating to 70° C. Once both phases are up to temperature, add Phase B to A and homogenise at 10,000 rpm for 2 minutes. Cool with stirring to below 40° C. before adding Phase C, allowing to mix well. Check pH and adjust as required.

### Example 12: Intensive Hair Mask

| INCI Name | % w/w |
|---|---|
| Aqua (Water) | Up to 100 |
| Glycerin | 1.50 |
| Hydroxyethylcellulose | 1.00 |
| Stearamidopropyl Dimethylamine | 1.00 |
| Citric Acid | 0.50 |
| Cetearyl Alcohol | 2.50 |
| Stearyl Alcohol | 2.50 |
| Elastomer Gel (A) Example 6 | 5.00 |
| Shorea Stenoptera Seed Butter | 0.50 |
| Behentrimonium Chloride (and) Isopropyl Alcohol | 0.75 |
| Preservative | q.s. |

| Appearance: | Opaque, white emulsion |
|---|---|
| Viscosity: | ~30,000 cP (Brookfield Helipath, T Bar C, 10 rpm) |
| pH: | 3.0-4.0 |

### Preparation Protocol:

Pour water into vessel and begin to heat. Premix Phase B and add to the water. The product should gel up around 50° C. Add Phase C and continue to stir. Add Phase D. Continue to heat to 80-85° C. Add Phase E. When at 80-85° C. and the fats have melted, homogenise at 4000 rpm until smooth. Cool to 40° C., add Phase F. Check viscosity and adjust pH if necessary.

### Example 13: Leave-In Hair Conditioner Formulation

| INCI Name | % w/w |
|---|---|
| Aqua (Water) | Up to 100 |
| Disodium EDTA | 0.02 |
| Sodium Benzoate | 0.40 |
| Polyquaternium-37 (and) Hydrogenated | 4.00 |
| Polydecene (and) Trideceth-6 (and) Aqua | |
| Citric Acid | 0.10 |
| Elastomer Gel (A) Example 6 | 5.00 |
| Stearyl Alcohol | 2.50 |

| Appearance: | White emulsion |
|---|---|
| Viscosity: | ~10,000 cP (Brookfield Helipath, T Bar C, 10 rpm) |
| pH: | 4.40-4.90 |

### Preparation Protocol:

Add water to the vessel and begin to stir. Add Phase A to the vessel and stir until a cream is formed. Homogenisation may be required. Add Phase B to the vessel and stir until homogenous. Further homogenisation may be required. Add Phase C with stirring. Check viscosity and pH and adjust if necessary.

### Application Tests

The hair conditioner formulations of Example 9 and 10 were tested as follows.

The reduction in hair friction was evaluated by measuring the coefficient of friction using the Dia-Stron MTT175 Miniature Tensile Tester on frizzy European hair tresses.

FIGS. 3 and 4 show the effect of the polyester elastomer component (A) on the percent improvement of friction on frizzy European hair tresses. FIGS. 3-4 show that the 10% polyester elastomer component (A) improves smoothing and damage repair on dry hair compared to conditioner bases which do not contain the polyester elastomer component (A). The results in FIGS. 3-4 were generated using a Dia-Stron MTT175 using a friction attachment. A hair tress is mounted flat on a plate, a rubber probe then moves across the hair tress to give a measurement of coefficient of friction. When the hair is moving with the cuticle, it is a measurement of smoothing, against the cuticle is damage repair. Measurements are taken prior to treatment−after washing with non-conditioning shampoo (baseline), and then again after treatment with the test products. Reduction in friction is indicated by a higher % improvement when comparing baseline to treated.

FIG. 5 and FIG. 6 show the anti-frizz effects of the hair using a Bossa Nova Vision Bolero.

Photos are taken prior to treatment—after washing with non-conditioning shampoo (baseline), then again after washing with the test products, at T=0, 1, 2, 4, 6, 24 and 48 hours. After washing with the test products, the tresses are dried overnight under controlled conditions, the T=0 image is taken, then the tresses are stored at 70% RH for the remainder of the test. Each image is processed by the Bolero software, the treated images are compared with baseline and the percentage change of the bulk surface from baseline is calculated. The software measures the area in cm² of bulk of the tress, i.e. the area of the hair without the frizz & flyaway fibres (the fibres protruding from the tress). The photos in FIGS. 5-6 are representative photos, which show the curl definition (and therefore less frizz) of each tress at the different time points.

1. A hair care composition comprising:
(A) at least one crosslinked polyester component, wherein the at least one crosslinked polyester component comprises a crosslinked polyester which is a reaction product of:
(i) at least one compound selected from the group consisting of poly-carboxylic acids poly-carboxylic acid esters, and combinations thereof, and
(ii) at least one polyol.

2. A The hair care composition of claim 1,
wherein the crosslinked polyester of (A) is further the reaction product of:
(iii) at least one mono-carboxylic acid; or
(iii) at least one mono-carboxylic acid and (iv) one or more mono-alcohol.

3. The hair care composition of claim 1, further comprising:
(C) at least one cosmetically acceptable carrier different from component (A).

4. The hair care composition of claim 1, wherein the hair care composition is selected from the group consisting of shampoo, conditioning shampoo, reconstructive shampoo, scalp care compositions, hair conditioning compositions, hair frizz controlling compositions, hair smoothing compositions, dry hair smoothing compositions, hair compositions, hair friction reducing compositions, hair combing facilitating compositions, hair repairing compositions, hair split ends reducing compositions, hair volume controlling compositions, hair volume-reducing compositions, bulk surface-reducing compositions, hair thickness-increasing compositions, hair strengthening compositions, hair colour protection compositions, hair shine enhancing compositions, hair moisturizing and hydrating compositions, hair deep conditioning compositions, hair hold conditioning compositions, hair curl defining compositions, hair cleansing conditioning compositions, leave-in conditioners, rinse-out conditioners, rinse-through conditioners, hair styling compositions, hair sprays, hair gels, hair creams, hair mousses, hair lotions, hair pastes, hair pomades, hair waxes, hair foams, and hair masks.

5. (canceled)

6. The hair care composition of claim 3, further comprising one or more hair care active compound (B), different from components (A) and (C), wherein the one or more hair care active compound (B) is selected from the group consisting of moisturizing agents, humectants, polyols, glycerin, propylene glycol, butylene glycol, sugar alcohols, sorbitol, panthenol, hyaluronic acid, Aloe Vera extract, honey, urea, conditioning agents, cationic surfactants, behentrimonium chloride, and cetrimonium chloride, silicones, dimethicone, cyclopentasiloxane, amodimethicone, polyquaternium-7, polyquaternium-10, stearalkonium chloride, proteins, such as hydrolyzed keratin, hydrolyzed silk protein, hydrolyzed collagen, wheat protein, soy protein, rice protein, lipids, or fatty acids, ceramides (Ceramide 1, Ceramide 2, Ceramide 3), fatty alcohols, shea butter, coconut oil, scalp treatment ingredients, salicylic acid, zinc pyrithione, tea tree oil, ketoconazole, niacinamide (vitamin B3), menthol, biotin (Vitamin B7), panthenol, ginseng extract, hair growth and repair agents, minoxidil, caffeine, finasteride, saw palmetto, copper peptides, argan oil, procapil, apple stem cells, antioxidants, vitamin E (Tocopherol), vitamin C, green tea extract, ferulic acid, resveratrol, UV protectants, benzophenone-4, ethylhexyl methoxycinnamate, homosalate, tinosorb S, dandruff treatment compounds, pyrithione zinc, tar extract, selenium sulfide, climbazole, volumizing agents, polyquaternium-11, panthenol, hydrolyzed wheat protein, anti-frizz agents, dimethicone, amodimethicone, polyquaternium-10, Triticum vulgare (wheat) germ oil, and chelating agents, ethylene diamine tetraacetic acid), and citric acid.

7. The hair care composition of claim 3, wherein the at least one cosmetically acceptable carrier (C) are selected from the group consisting of water, solvents, emollients, fatty acids, fatty alcohols, film formers, emulsifiers, thickeners and combinations thereof.

8. (canceled)

9. The hair care composition of claim 1, wherein
the at least one compound (i) is selected from the group consisting of
(1) one or more poly-carboxylic acids of formula (I) 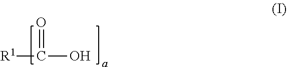
wherein
R¹ is selected from the group consisting of C2-C52 alkyl group, C2-C52 heteroalkyl group, C2-C52 alkene group, C2-C52 heteroalkene group, C3-C52 cyclic group, and C2-C52 heterocyclic group, and
a is an integer from 2 to 10,
(2) one or more poly-carboxylic acid esters of formula (II) 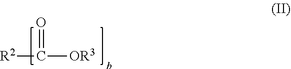
wherein
R² is C1-C22 alkyl group, C2-C22 alkylene group, or C3-C22 cyclic group;
R³ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
b is an integer from 2 to 10, and
(3) a combination thereof,
the at least one polyol (ii) is a polyol of formula (III) 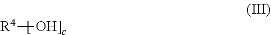
wherein
R⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
c is an integer from 2 to 10.

10. The hair care composition of claim 1, wherein
the at least one compound (i) is one or more poly-carboxylic acids is selected from the group consisting of di-carboxylic acids, tri-carboxylic acids; and combinations thereof,
the one or more polyol (ii) is selected from the group consisting of diols, triols, and combinations thereof.

11. (canceled)

12. The hair care composition of claim 1, wherein the at least one crosslinked polyester component (A) has a gel fraction which is greater than 20%.

13. The hair care composition of claim 1, wherein the at least one crosslinked polyester component (A) has a swelling ratio of from about 1 gram/gram to about 15 gram/gram.

14. The hair care composition of claim 1, wherein the at least one crosslinked polyester component (A) is comprised of particles of a size from about 1 μm to about 500 μm as measured by a laser diffraction particle size analyzer.

15. The hair care composition of claim 1, wherein the at least one crosslinked polyester component (A) is obtained by:
(a) reacting the at least one compound (i) and the at least one polyol (ii)
to obtain the crosslinked polyester component of (A);
and
(b) subjecting the crosslinked polyester component of (A) obtained in (a) to shear force to obtain the cross-linked polyester component (A) as a gel; or
(c) adding one or more solvents, one or more emollients, or a combination thereof to the crosslinked polyester component of (A) obtained in (a), allowing the resulting mixture to swell in the one or more solvents, one or more emollients, or a combination thereof and subjecting the swollen mixture to shear force to obtain the at least one cross-linked polyester component (A) as a gel.

16. (canceled)

17. The hair care composition claim 15, wherein shear force in (b) and (c) is carried out with a batch mixer, planetary mixer, single or multiple screw extruder, dynamic or static mixer, colloid mill, homogenizer, sonolator, three roll mill, or a combination thereof.

18. (canceled)

19. The hair care composition of claim 15, wherein the amount of the one or more solvents, one or more emollients, or a combination thereof added in (c) is such that the resulting at least one crosslinked polyester component (A) comprises from about 10% to about 95% weight by weight of the one or more solvents, one or more emollients, or a combination thereof.

20. The hair care composition of claim 3, wherein the at least one crosslinked polyester component (A) is provided as a gel, and wherein the at least one cosmetically acceptable carrier (C) is a triglyceride solvent, a mono-ester solvent, a di-ester solvent, a citrate ester solvent, an ether solvent, a carbonate solvent, a hydrocarbon solvent, a silicone solvent, or a combination thereof.

21. The hair care composition of claim 3, further comprising one or more other components commonly used in the cosmetic field (D), different from components (A), (B), or (C), wherein the one or more other components commonly used in the cosmetic field (D) are selected from the group consisting of UV filter compounds, suspending agents, surfactants, emulsifiers, preservatives, rheology modifiers, pH adjustors, reducing agents, anti-oxidants, foaming agents, de-foaming agents, chelating agents, gums or thickeners, oils, waxes, fragrances, essential oils, and combinations thereof.

22. The hair care composition of claim 3, comprising:
0.1 to 85% by weight of at least one crosslinked polyester component (A),
0 to 80% by weight of one or more hair care active compounds (B),
0 to 90% by weight of at least one cosmetically acceptable carrier (C), and
0 to 60% by weight of one or more other components commonly used in the cosmetic field (D),
based on the total weight of the composition.

23. The hair care composition of claim 3, consisting of:
(A) at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol; and
(C) at least one cosmetically acceptance carrier, wherein (C) is different from component (A): or
(A) at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol;
(B) one or more hair care active compounds, wherein (B) is different from components (A) and (C); and
(C) at least one cosmetically acceptable carrier, wherein (C) is different from components (A) and (B).

24.-29. (canceled)

30. The hair care composition of claim 2, wherein:
the at least one mono-carboxylic acid (iii) is a mono-carboxylic acid of formula (IV) 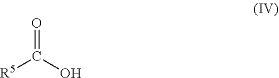
wherein
R⁵ is C2-C52 alkyl group, C2-C52 heteroalkyl group, C2-C52 alkene group, C2-C52 heteroalkene group, C3-C52 cyclic group, or C2-C52 heterocyclic group; and
the one or more mono-alcohol of (iv) is a mono-alcohol of formula (V) 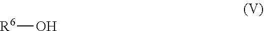
wherein
R⁶ is C2-C52 alkyl group, C2-C52 heteroalkyl group, C2-C52 alkene group, C2-C52 heteroalkene group, C3-C52 cyclic group, or C2-C52 heterocyclic group.
